SEQUENCE LISTING (4 sequences)

SEQ ID NO: 1 - protein, 313 aa, synthetic construct
  source 1..313: note = fusion protein
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGSGGGYGRKKRRQRRR

SEQ ID NO: 2 - protein, 11 aa, synthetic construct
  source 1..11: note = tat polypeptide
YGRKKRRQRRR

SEQ ID NO: 3 - protein, 14 aa, synthetic construct
  source 1..14: note = MGGSHHHHHHGMAS
MGGSHHHHHHGMAS

SEQ ID NO: 4 - protein, 302 aa, synthetic construct
  source 1..302: note = FMRP(289)
MEELVVEVRGSNGAFYKAFVKDVHEDSITVAFENNWQPDRQIPFHDVRFPPPVGYNKDINESDEVEVYSRANEKEPCCWWLAKVRMIKGEFYVIEYAACDATYNEIVTIERLRSVNPNKPATKDTFHKIKLDVPEDLRQMCAKEAAHKDFKKAVGAFSVTYDPENYQLVILSINEVTSKRAHMLIDMHFRSLRTKLSLIMRNEEASKQLESSRQLASRFHEQFIVREDLMGLAIGTHGANIQQARKVPGVTAIDLDEDTCTFHIYGEDQDAVKKARSFLEFAEDVIQVPRNLVGKVIGSGGG

(19) United States
(12) Patent Application Publication
(10) Pub. No.: US 2025/0026798 A1
(43) Pub. Date: Jan. 23, 2025

(54) TREATMENT OF FRAGILE X SYNDROME

(71) Applicant: UTI Limited Partnership, Calgary (CA)

(72) Inventor: Raymond W. TURNER, Calgary (CA)

(21) Appl. No.: 18/906,893

(22) Filed: Oct. 4, 2024

Related U.S. Application Data
(63) Continuation of application No. 18/167,010, filed on Feb. 9, 2023, now abandoned.
Application No. 18/167,010 is a continuation of application No. 16/961,345, filed on Jul. 10, 2020, now abandoned.
(60) Provisional application No. 62/616,140, filed on Jan. 11, 2018.

(51) Int. Cl. C07K 14/435; A61K 31/155; A61K 31/351; A61K 31/65; A61K 38/00; A61P 43/00; C07K 14/005

(52) U.S. Cl. CPC C07K 14/435; A61K 31/155; A61K 31/351; A61K 31/65; A61P 43/00; C07K 14/005; A61K 38/00; C07K 2319/21; C07K 2319/71; C07K 2319/85

(57) ABSTRACT

The present disclosure relates generally to the treatment of Fragile X Syndrome using a recombinant fusion polypeptide comprising or consisting of a cell penetrating polypeptide, such as HIS or tat, and a Fragile X Mental Retardation protein (FMRP (298)).

### SEQUENCE LISTING

The instant application contains a Sequence Listing which has been submitted electronically in .xml format and is hereby incorporated by reference in its entirety. Said .xml copy, created on Oct. 4, 2024, is named “51012-034003_Sequence_Listing_10_4_24.xml” and is 5,650 bytes in size.

### FIELD

The present disclosure relates generally to the treatment of Fragile X Syndrome.

### BACKGROUND

Fragile X Syndrome is the most common genetic cause of intellectual disability and incidence of autism spectrum disorder. Fragile X results from an inordinate number of CGG repeats (>200) on the UTR region of the fmrp1 gene on the X chromosome that leads to hypermethylation and block of transcription of the fmrp1 gene. As a result, there is a loss of expression of Fragile X Mental Retardation Protein (FMRP) that is known to regulate translation or activity of multiple proteins required to exhibit normal levels of synaptic plasticity¹⁻¹⁰. The fact that this disorder arises from the loss of a single protein provides an incentive to understand how it disrupts synaptic plasticity and to identify a treatment strategy that either restores FMRP or blocks secondary adverse events in order to reduce behavioural dysfunctions of Fragile X syndrome. Central to these tests is extensive use of a FMRP−/− mouse line that effectively recapitulates the key genetic disruption of FMRP expression, with many traits similar to that of Fragile X patients that harbor the full genetic mutation.

### SUMMARY

In one aspect there is described a recombinant fusion polypeptide comprising or consisting of a cell penetrating polypeptide and a FMRP(298) polypeptide, or fragment or variants thereof.

In one example, said cell penetrating polypeptide comprises a tat polypeptide.

In one example, the said tat polypeptide comprises YGRKKRRQRRR (SEQ ID NO: 2).

In one example, the further comprising a HIS polypeptide.

In one example, the said HIS polypeptide comprises MGGSHHHHHHGMAS (SEQ ID NO: 3).

In one aspect there is described a fusion polypeptide comprising or consisting of tat-FMRP(298) MEELVVEVRGSNGAFYKAFVKDVHEDSITVAFENNWQPDRQIPFHDVRFPPPVGYNKDINESD EVEVYSRANEKEPCCWWLAKVRMIKGEFYVIEYAACDATYNEIVTIERLRSVNPNKPATKDTFH KIKLDVPEDLRQMCAKEAAHKDFKKAVGAFSVTYDPENYQLVILSINEVTSKRAHMLIDMHFRSL RTKLSLIMRNEEASKQLESSRQLASRFHEQFIVREDLMGLAIGTHGANIQQARKVPGVTAIDLDE DTCTFHIYGEDQDAVKKARSFLEFAEDVIQVPRNLVGKVIGSGGGYGRKKRRQRRR (SEQ ID NO: 1), or fragments or variants thereof.

In one example, the said fusion polypeptide comprises a variant fusion polypeptide sequence that is at least 80-99% identical to said fusion polypeptide, or fragments or variants thereof.

In one example, the recombinant fusion polypeptide having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70,71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more than 100 amino acid substitutions.

In one aspect there is described a polynucleotide molecule comprising or consisting of a sequence that encodes a cell penetrating polypeptide and a FMRP(298) polypeptide, or fragment or variants thereof. In one example, the said cell penetrating polypeptide comprises a tat polypeptide.

In one example, the said tat polypeptide comprises YGRKKRRQRRR (SEQ ID NO: 2).

In one aspect there is described a polynucleotide molecule comprising or consisting of a sequence that encodes a fusion polypeptide comprising or consisting of tat-FMRP(298) (SEQ ID NO: 1).

In one aspect there is described a polynucleotide molecule comprising or consisting of a sequence that encodes a fusion polypeptide according to any one of claims 1 to 11.

In one aspect there is described a vector comprising the polynucleotide molecule of any one of claims 9 to 13.

In one aspect there is described a mammalian cell comprising the polynucleotide molecule of any one of claims 9 to 13.

In one aspect there is described a mammalian cell comprising the vector of claim 14.

In one aspect there is described a pharmaceutical composition comprising a recombinant fusion polypeptide of any one of claims 1-8, and a pharmaceutically acceptable carrier.

In one aspect there is described a method of treatment of a subject having or suspected of having Fragile X Syndrome, comprising: administering a recombinant fusion polypeptide of any one of claims 1 to 8, or a pharmaceutical composition of claim 17, to said subject.

In one example, further comprising administration of minocycline, metformin, and/or blockers of extracellular signal-regulated kinase (ERK).

In one example, the said subject is a human.

In one aspect there is described a use of a recombinant fusion polypeptide of any one of claims 1 to 8, or a pharmaceutical composition of claim 17, for the treatment of a subject having or suspected of having Fragile X Syndrome.

In one example, further comprising the use of minocycline, metformin, and/or blockers of extracellular signal-regulated kinase (ERK) such as lovastatin, for the treatment of a subject having or suspected of having Fragile X Syndrome.

In one aspect there is described a use of a recombinant fusion polypeptide of any one of claims 1 to 8, or a pharmaceutical composition of claim 17, in the manufacture of a medicament for the treatment of a subject having or suspected of having Fragile X Syndrome.

In one example, the further comprising further comprising the use of minocycline, metformin, and/or blockers of extracellular signal-regulated kinase (ERK) such as lovastatin, in the manufacture of medicament for the treatment of a subject having or suspected of having Fragile X Syndrome.

In one example, the use of any one of claim 18 or 24, wherein said subject is a human.

In one aspect there is described a kit, comprising: a container; a recombinant fusion polypeptide of any one claims 1 to 8, and/or a polynucleotide of any one of claims 9 to 13, and/or a vector of claim 14, a mammalian cell of claim 15 or 16, and/or a pharmaceutical composition of claim 17; and optionally instructions for the use thereof.

### BRIEF DESCRIPTION OF THE FIGURES

Embodiments of the present disclosure will now be described, by way of example only, with reference to the attached Figures.

FIGS. 1A-1C. A Cav3-Kv4 complex is modified in long-term potentiation. Theta burst stimulation (TBS) of mossy fibers to induce LTP left shifts Kv4 Vh to reduce A-type K+ current (FIG. 1A), potentiates the mossy fiber-evoked EPSP (FIG. 1B), and increases intrinsic excitability and spike firing in granule cells (FIG. 1C).

FIGS. 2A-2B. LTP of mossy fiber input (TBS) reduces A-type current by shifting Cav3-Kv4 Vh in wild type (FIG. 2A) but not FMRP−/− mice (FIG. 2B). Insets show A-type current for a step from −70 to −30 mV.

FIGS. 3A-3D. FIG. 3A, Infusing 35 nM FMRP(298) in tsA-201 cells expressing Cav3.1 induces a leftward shift in Cav3.1 Vh and a decrease in T-type current. FIG. 3B, Infusing 3 nM FMRP(298) into FMRP−/− granule cells iduces a left shift in Kv4 Vh and a reduction in A-type current. Insets show currents evoked by a step from −70 mV to −30 mV. FIG. 3C, FMRP coIPs with Cav3.1 from cerebellar lysates. FIG. 3D, Coexpressing GFP-Cav3.1 and mKate-FMRP(298) reveals FRET as an emission by mKate at 660 nm in response to 457 laser excitation of GFP.

FIGS. 4A-4C. Mossy fiber LTP is FMRP-dependent. Mossy fiber EPSP amplitude in granule cells be-fore and after TBS (arrow) in wild type (FIG. 4A), FMRP−/− mice (FIG. 4B), and FMRP−/− with 3 nM FMRP(298) in the electrode (FIG. 4C). Spike firing is noted above and the X-axis is truncated for 5 min when no stimuli are applied.

FIGS. 5A-5B. FIG. 5A, Perfusing 100 pM tat-FMRP(298) left-shifts Cav3.1 Vh expressed in isolation in tsA-201 cells. FIG. 5B, Perfusing 100 pM tat-FMRP(298) left-shifts the Cav3-Kv4 Vh in granule cells of FMRP−/− mice. Insets show the effects of tat-FMRP(298) infusion on Cav3.1 (FIG. 5A) and the Kv4 current recorded in granule cells (FIG. 5B) under conditions in which Cav3 current is intact to gauge its effects on the Cav3-Kv4 complex.

FIGS. 6A-6C. FMRP−/− mice differ from wild type animals on a battery of behavioural tests. FIG. 6A, FMRP−/− animals exhibit hallmark signs of hyperactivity in an Open Field test compared to wt animals. FIG. 6B, FMRP−/− mice show a much higher level of dominance in a Tube Test. FIG. 6C, FMRP−/− show less grip force as a measure of cerebellar and motor control functions.

FIGS. 7A-7D. FIGS. 7A-7B, FMRP immunolabel is present in granule cells of all lobules, shown in a rat cerebellar sagital section. FIGS. 7C-7D, FMRP label is lacking in FMRP−/− mice (FIG. 7C) but present 1 hr after tail vein injection of 100 nM tat-FMRP(298) (FIG. 7D).

FIGS. 8A-8E. Track path plots in the Open Field test (FIG. 8A, FIG. 8B) and the frequency of center crosses (FIG. 8E) reveals hyperactivity in FMRP−/− compared to wt mice. FIGS. 8C-8D, Hyperactivity is little affected by tail vein injections of vehicle (FIG. 8C) but significantly reduced after tat-FMRP(298) injection (FIG. 8D, FIG. 8E). Sample numbers are indicated in brackets.

FIGS. 9A-9I. Mossy fiber LTP and modulation of the Cav3-Kv4 complex depends on FMRP. FIGS. 9A-9C, Plots of the mean amplitude of the mossy fiber-evoked EPSP and spike occurrence per stimulus measured in whole-cell recordings of lobule 9 granule cells. EPSP amplitudes were only calculated for stimuli that were subthreshold to spike discharge. FIGS. 9A-9B, Theta burst stimulation (TBS) of mossy fiber input evokes LTP of the fiber EPSP and an increase in probability of firing in wt (FIG. 9A) but not Fmr1−/y mice (FIG. 9B). (FIG. 9C), Infusing 3 nM FMRP(1-297) into granule cells rescues LTP of spike output in Fmr1−/y mice. FIGS. 9D-9I, Plots of the voltage for inactivation and activation of Kv4 current in granule cells in resting conditions (control) and following TBS of mossy fiber input. Insets in (FIG. 9D, FIG. 9F, FIG. 9H) superimpose Kv4 current evoked by a step from −70 mV to −30 mV for either condition. FIGS. 9D-9E, Following TBS Kv4 Vh and Va are left-shifted in wt mice (FIG. 9D, FIG. 9E) but not in Fmr1−/y mice (FIG. 9F, FIG. 9G). (FIGS. 9H-9I, Infusing 3 nM FMRP(1-297) into granule cells of Fmr1−/y mice restores the ability for TBS stimulation to left shift Kv4 Vh and Va to reduce Kv4 current amplitude. Average values are mean±SEM; * p<0.05; ** p<0.01; *** p<0.001, Students t-test.

FIGS. 10A-10D. tat-FMRP(1-298) exhibits a concentration-dependent effect on hyperactivity in FMRP KO mice. Shown are bar plots of the effects of tail vein injecting tat-FMRP(1-298) at the indicated concentrations on different aspects of movement in an open field test cage 1 hr (FIG. 10A, FIG. 10C) or 24 hrs (FIG. 10B, FIG. 10D) following injections. FMRP KO mice are significantly hyperactive compared to wild type (wt) animals in measures of either Total Distance traveled or Velocity in the Center zone. A significant concentration-dependent effect of tat-FMRP(1-298) is detected for 100 nM and 500 nM but not for 1 μM for both total distance traveled (FIG. 10A, FIG. 10B) and Velocity in the cage center (FIG. 10C, FIG. 10D) 1 hr and 24 hrs after injection. No effects are apparent for injecting 100 nM tat-FMRP(1-298) in wild type animals either 1 hr (FIG. 10C, FIG. 10D) or 24 hrs (FIG. 10B, FIG. 10D) after injections. Average values are mean±SEM; * p<0.05; ** p<0.01; *** p<0.001, Students t-test.

FIGS. 11A-11D. tat-FMRP(1-298) injections invoke a concentration-dependent recovery of LTP at the mossy fiber synapse of FMRP KO mice. FIG. 11A, Schematic of tail vein injections of different concentrations of tat-FMRP(1-298) followed 1 hr later by preparation of cerebellar tissue slices to conduct recordings from granule cells. FIG. 11B, The effects of injecting 500 nM tat-FMRP(1-298) on mossy fiber evoked theta burst stimulation (TBS), with full recovery of EPSP amplitude and spike changes expected in a wild type animal. FIG. 11C, Comparison of the effects of direct infustion of 3 nM tat-FMRP(1-298) on TBS-evoked granule cell responses where primary changes are only detected in spike firing rate. FIG. 11D, Tail vein injection of 100 nM tat-FMRP(1-298) does not invoke recovery of LTP at the mossy fiber synapse when tested in vitro.

FIGS. 12A-12B. tat-FMRP(1-298) applied directly to cerebellar granule cell cultures is not toxic up to 5 days in culture. FIG. 12A, Flow cytometric analysis of labeling for a live-dead cell viability assay for Cy5 and FITC markers in dissociated mouse cerebellar granule cell cultures 24 hrs after a single exposure to 500 nM tat-FMRP(1-298). The proportion of Live to Dead cells identified by flow cytometry does not differ between cells left untreated, treated with vehicle alone, or with tat-FMRP(1-298). FIG. 12B, Bar plots of mean cell counts for the identified concentrations of a single dose of vehicle or tat-FMRP(1-298) tested at 24 hrs and 5 days after treatment and measured by flow cytometry.

FIGS. 13A-13C. tat-FMRP(1-298) reduces gamma frequencies in EEG recordings. FIG. 13A, FIG. 13B, Shown are average frequency spectrum plots for EEG recorded for 30 min 24 hrs following tail vein injections of vehicle alone (FIG. 13A) or 500 nM tat-FMRP(1-298) (FIG. 13B) in wt or FMRP KO mice. FRMP KO mice show elevated levels of gamma frequency activity (demarked for frequencies above 40 Hz in FIG. 13A) (adapted from

Lovelance et al. (2018). FIG. 13B, FIG. 13C, Spectral power density plots of EEG recorded from FMRP KO or wt mice using skull surface EEG electrodes differentially recording between a cortical and cerebellar-located electrode. FIG. 13B, Animals vehicle injected show a baseline different in higher frequency EEG activity, as reported by Lovelace et al. (2018). FIG. 13C, 500 nM tat-FMRP(1-298) injection reduces gamma frequency activities (red arrows) even 24 hrs post tail vein injections.

### DETAILED DESCRIPTION

Generally, the present disclosure relates to the treatment of Fragile X syndrome.

Fragile X syndrome (FXS) refers to a genetic disease associated with and/or caused by to a defect of the expression of the FMR1 gene and/or of the activity of the FMR1-encoded polypeptide, FMRP.

In some examples, signs and symptoms of FXS may fall into five categories: intelligence and learning; physical, social and emotional, speech and language and sensory disorders commonly associated or sharing features with Fragile X.

For example, individuals with FXS may have impaired intellectual functioning, social anxiety, language difficulties and sensitivity to certain sensations.

Cognitive disorders may include, but are not limited to, the group of disorders in which a dysfunction/impairment of mental processing constitutes the core symptomatology. Cognitive disorders include neurogenetic cognitive disorders or behavioral cognitive disorders

Cognitive disorders may include, but are not limited to, developmental disorders, attention deficit hyperactivity disorder (ADHD), autism spectrum disorders, Alzheimers disease, schizophrenia and cerebrovascular disease.

Autism spectrum disorders and autistic symptoms are commonly associated with individuals with Fragile X syndrome. Signs and symptoms of autism may include, but are not limited to, significant language delays, social and communication challenges, and unusual behaviors and interests. Individuals with autistic disorder may also have intellectual disability.

Methods of assessment of Fragile X Syndrome in a subject are known. Accordingly, methods of assessing efficacy of treatment of Fragile X Syndrome in a subject are known.

In one aspect, there is provided a fusion polypeptide comprising or consisting of a cell penetrating polypeptide and a FMRP(298) polypeptide, for the treatment of a subject having or suspected of having Fragile X Syndrome.

In one example, the cell penetrating polypeptide may be located at the N-terminus of the fusion polypeptide. In one example, the cell penetrating polypeptide may be located at the C-terminus of the fusion polypeptide. In one example, the cell penetrating polypeptide may be located in an internal location of the fusion protein.

In one example, the fusion polypeptide comprises a HIS polypeptide. In a specific example, the HIS peptide is MGGSHHHHHHGMAS (SEQ ID NO: 3)

In a specific example, the cell penetrating polypeptide comprises or consists of a tat polypeptide. In a specific example, the tat polypeptide is YGRKKRRQRRR (SEQ ID NO: 2).

In one example, the FMRP(298) sequence is

(SEQ ID NO: 4)
MEELVVEVRGSNGAFYKAFVKDVHEDSITVAFENNWQPDRQIPFHDVRF
PPPVGYNKDINESDEVEVYSRANEKEPCCWWLAKVRMIKGEFYVIEYAA
CDATYNEIVTIERLRSVNPNKPATKDTFHKIKLDVPEDLRQMCAKEAAH
KDFKKAVGAFSVTYDPENYQLVILSINEVTSKRAHMLIDMHFRSLRTKL
SLIMRNEEASKQLESSRQLASRFHEQFIVREDLMGLAIGTHGANIQQAR
KVPGVTAIDLDEDTCTFHIYGEDQDAVKKARSFLEFAEDVIQVPRNLVG
KVIGSGGG

In a specific example, there is provided a fusion polypeptide comprising or consisting of tat-FMRP(298) (MGGSHHHHHHGMASMEELVVEVRGSNGAFYKAFVKDVHEDSITVAFENNWQPDRQIPFHDV RFPPPVGYNKDINESDEVEVYSRANEKEPCCWWLAKVRMIKGEFYVIEYAACDATYNEIVTIER LRSVNPNKPATKDTFHKIKLDVPEDLRQMCAKEAAHKDFKKAVGAFSVTYDPENYQLVILSINE VTSKRAHMLIDMHFRSLRTKLSLIMRNEEASKQLESSRQLASRFHEQFIVREDLMGLAIGTHGA NIQQARKVPGVTAIDLDEDTCTFHIYGEDQDAVKKARSFLEFAEDVIQVPRNLVGKVIGSGGGY GRKKRRQRRR) (SEQ ID NO: 1), for the treatment of a subject having or suspected of having Fragile X Syndrome

The term “subject” or “patient” are used synonymously, and as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. The subject may be an infant, a child, an adult, or elderly. In a specific example, the subject is a human.

As used herein, “treatment” refers to any manner in which one or more of the symptoms of a disorder, such as FXS, are ameliorated or otherwise beneficially altered. Thus, the terms “treating” or “treatment” of a disorder as used herein includes: reverting the disorder, i.e., causing regression of the disorder or its clinical symptoms wholly or partially; preventing the disorder, i.e. causing the clinical symptoms of the disorder not to develop in a subject that can be exposed to or predisposed to the disorder but does not yet experience or display symptoms of the disorder; inhibiting the disorder, i.e., arresting or reducing the development of the disorder or its clinical symptoms; attenuating the disorder, i.e., weakening or reducing the severity or duration of a disorder or its clinical symptoms; or relieving the disorder, i.e., causing regression of the disorder or its clinical symptoms. Further, amelioration of the symptoms of a particular disorder by administration of a particular composition refers to any lessening, whether permanent or temporary, lasting or transient that can be attributed to or associated with administration of the disclosed compounds, compositions, etc.

As used herein, the terms “polypeptide”, “peptide” and “protein,” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

In some examples, a “fusion polypeptide” or “fusion protein” is a recombinant protein of two or more polypeptides which are joined by a peptide bond. In some examples, the two or more polypeptides may be joined by a linker.

The fusion polypeptide may include variants of a fusion polypeptide. In some examples, a variant of a fusion polypeptide refers to fusion polypeptides having different sequence from wild type amino acid sequence. For examples, a variant fusion polypeptide may have deletions, insertions, non-conservative or conservative substitutions of at least one amino acid residue, or combinations thereof.

In some example, the recombinant polypeptide is a variant of the polypeptide of the recombinant fusion protein tat-FMRP(298).

In some examples, the “variant” are it relates to polypeptides refers to polypeptides having an amino acid sequence that is at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% or greater identical to the parental amino acid sequence.

The fusion polypeptide and/or variants thereof may be chemically synthesized or produced by gene recombination, and it may be produced by transforming host cells using a recombinant vector and separating and purifying expressed protein.

The term “recombinant” as used herein refers to a non-naturally occurring nucleic acid, nucleic acid construct, or polypeptide. Such non-naturally occurring nucleic acids can include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a “fusion protein” (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter etc.). Recombinant also refers to the polypeptide encoded by the recombinant nucleic acid. Recombinant may also refers to refer to a polypeptide or polynucleotide, for example, that is no longer in its natural environment

Also provided herein are recombinant polynucleotides that may encode one or more of the recombinant fusion polypeptides described herein. In one example, a polynucleotide encodes a polypeptide comprising or consisting of the recombinant fusion protein tat-FMRP(298).

A polynucleotide encoding the fusion protein may be codon optimized for efficient translation into a polypeptide in the eukaryotic cell or animal of interest.

As used herein the terms “polynucleotide” and “nucleic acid’ refer to two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. Typically nucleosides will be linked together via standard phosphodiester linkages. However, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine, and cytosine), it may include one or more modified and/or synthetic nucleobases (e.g., inosine, xanthine, hypoxanthine, etc.). Polynucleotide includes, but is not limited to chemically, enzymatically, or metabolically modified forms.

In some examples there is provided a polynucleotide molecule comprising or consisting of a sequence that encodes a tat-FMRP(298) fusion polypeptide.

In some examples, there is provided a variant of a polynucleotide molecule comprising or consisting of a sequence that encodes a tat-FMRP(298) fusion polypeptide.

As used herein, the terms “polynucleotide variant” and the like refer to polynucleotides displaying substantial sequence identity with a reference polynucleotide sequence or polynucleotides that hybridize with a reference sequence under, for example, stringent conditions. These terms may include polynucleotides in which one or more nucleotides have been added or deleted, or replaced with different nucleotides compared to a reference polynucleotide. It will be understood that that certain alterations inclusive of mutations, additions, deletions and substitutions can be made to a reference polynucleotide whereby the altered polynucleotide retains the biological function or activity of the reference polynucleotide.

In some examples, the “variant” as it relates to polynucleotides refers to polynucleotides having an nucleotide sequence that is at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% or greater identical to the parental polynucleotide sequence.

In some examples, the recombinant fusion polypeptide is encoded by a variant fusion polynucleotide that binds under high hybridization stringency to the fusion polynucleotide.

As used herein the term “hybridization stringency” refers to hybridization conditions, such as washing conditions, in the hybridization of nucleic acids. Generally, hybridization reactions are performed under conditions of lower stringency, followed by washes of varying but higher stringency.

Under high stringency conditions, a polynucleotide with higher identity is expected to hybridize efficiently at higher temperatures, though multiple factors are involved in hybridization stringency including temperature, probe concentration, probe length, ionic strength, time, salt concentration and others, and a person skilled in the art may appropriately select these factors to achieve similar stringency.

In some examples “high stringency” may refer to the use of a hybridization or wash solution comprising 10 mM phosphate buffer, pH 7.0, at a range of about 45-55° C. In some examples, “moderate stringency” may refer to the use of 10 mM phosphate buffer, pH 7.0, with a salt concentration of about 0.1 to 0.5 M NaCl, at a temperature of between about 30 to 45° C. In some examples, “low stringency” may refer to the use of about 10 mM phosphate buffer at about pH 7.0, 1.0 M NaCl at room temperature. Low stringency buffers may also include 10 mM MgCl2. It will be understood that that many factors, such as temperature, salt and inclusion of other components such as formamide, affect the stringency of hybridization.

Under high stringency conditions, a polynucleotide with higher identity is expected to hybridize efficiently at higher temperatures, though multiple factors are involved in hybridization stringency including temperature, probe concentration, probe length, ionic strength, time, salt concentration and others, and a person skilled in the art may appropriately select these factors to achieve similar stringency.

In one example, there is provided a vector comprising a polynucleotide as described herein.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host ceil DNA. The polypeptide or polynucleotide may be isolated.

By an “isolated” polypeptide, polynucleotide, fragment, variant, or derivative thereof is i is not in its natural milieu. No particular level of purification is required. For example, an isolated polypeptide can be removed from its native or natural environment. Recombinantly produced polypeptides expressed in host cells are considered isolated for purposed of the invention, as are native or recombinant polypeptides which have been separated, fractionated, or partially or substantially purified by any suitable technique.

In some examples, the isolated polypeptide or polypeptide may be purified.

As used herein, “pure” or “purified” means an object species is the predominant species present (i.e., on a molar and/or mass basis, it is more abundant than any other individual species, apart from water, solvents, buffers, or other common components of an aqueous system in the composition), and, in some embodiments, a purified fraction is a composition wherein the object species comprises at least about 50% (on a molar basis) of all macromolecular species present. Generally, a “substantially pure” composition will comprise more than about 80% of all macromolecular species present in the composition, in some embodiments more than about 85%, more than about 90%, more than about 95%, or more than about 99%. In some embodiments, the object species is purified to essential homogeneity (contaminant species cannot be detected in the composition by conventional detection methods) wherein the composition consists essentially of a single macromolecular species.

In one example, the recombinant fusion polypeptide(s) described herein may be used for administration to a subject.

Administration may be in vitro, ex vivo or in vivo.

Administering may also be performed, for example, once, a plurality of times, and/or over one or more extended periods.

Administration may be by any suitable means.

In some examples, the recombinant polypeptides are formulated as a pharmaceutical composition, which is pharmaceutically acceptable.

The phrase “pharmaceutically acceptable” indicates that the substance or composition must be compatible chemically and/or toxicologically, with the other ingredients comprising a formulation, and/or the subject being treated.

The recombinant fusion polypeptide may be formulated with pharmaceutically acceptable carriers, excipients or diluents.

Pharmaceutically acceptable carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions as described herein may be sterilized by conventional methods and/or lyophilized.

Routes of administration include, but are not limited to, injection (subcutaneous, intravenous, parenterally, intraperitoneally, intrathecal), oral, inhalation, rectal and transdermal. The pharmaceutical compositions may be given by forms suitable for each administration route. For example, these compositions are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, a compound described herein can be coated with or disposed in a selected material to protect it from natural conditions which may detrimentally affect its ability to perform its intended function. A compound or composition described herein can be administered alone, or in conjunction with either another agent as described above or with a pharmaceutically-acceptable carrier, or both. A compound or composition described herein can be administered prior to the administration of the other agent, simultaneously with the agent, or after the administration of the agent. Furthermore, a compound described herein can also be administered in a pro-drug form which is converted into its active metabolite, or more active metabolite in vivo.

In some examples, there is further provided co-administration or use with a second agent. In some example, the second agent may be minocycline, metformin, and/or blockers of extracellular signal-regulated kinase (ERK) such as lovastatin and related compounds.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1

There is a growing initiative to restore FMRP transcription or pharmacologically intervene with down-stream effectors of FMRP¹¹⁻²². While promising, these often require genetic modifications at the embryonic stage or target molecules secondary to the loss of FMRP. An alternative strategy would be to use the HIV-1 regulatory protein, trans-activator of transcription (tat) peptide²³⁻²⁵ conjugated to FMRP to facilitate passage across membranes to gain intracellular access. We are thus taking the approach of reintroducing FMRP protein to FMRP−/− mice at defined levels using tat-peptide conjugates, and will test its effects at an identified cerebellar synaptic junction in vitro and in behavioural assays in vivo. Our data shows that directly in-fusing a short active fragment of the N terminal region of FMRP (aa 1-298) into cerebellar granule cells in vitro restores the function of an ion channel complex disrupted in FMRP−/− mice and reinstates the capacity for synaptic plasticity at the mossy fiber synapse. To implement this approach across a wide population of cells we designed a tat-FMRP(298) construct that also restores FMRP-induced modulation of Cav3 channels following bath application in vitro. Moreover, when injected into the tail vein of FMRP−/− animals tat-FMRP(298) (100 nM) rapidly crosses the blood brain barrier to enter neurons across the brain. Preliminary tests reveal its capacity to reduce the hyperactivity characteristic of Fragile X syndrome within 1 hour in even adult (P90) mice, with no obvious detriment to animal behaviour in terms of motor function, vocal communication, or socialization. Initial tests for toxicity have applied 100 nM tat-FMRP(298) directly to dissociated cultures of cerebellar granule cells for 24 hr, with no change in the percentage of cells labeled in a live-dead test kit compared to only saline vehicle.

These studies reveal a new function for FMRP in acting on a Cav3-Kv4 ion channel complex to regulate excitability and synaptic plasticity. They further reveal that reintroducing a short N-terminal fragment of FMRP as a tat-enabled peptide can restore synaptic plasticity and reduce behavioural symptoms in an animal model of Fragile X. We can thus assess the ability for tat-FMRP constructs to replace the very factor that is missing in Fragile X, with the advantage of measuring its influence on an identified ion channel complex involved in synaptic plasticity. Together these experiments will define the ability to use short tat-FMRP constructs as a therapeutic approach to reinstate FMRP function in cerebellum and other regions of the CNS to eventually treat this genetic disorder.

### Cerebellar Signal Processing in Fragile X Syndrome

The cerebellum is positioned above the hindbrain as a separate cortical structure comprised of three distinct layers of granule cells, Purkinje cells and an overlying molecular layer. The essential elements of circuit processing are sensory activation=>mossy fiber input=>granule cells=>parallel fiber input to Purkinje cells=>Purkinje cell output through deep nuclei to other brain regions. A key role for cerebellum is receiving a copy of all sensory input arriving from the periphery, as well as a copy of cortical motor commands for movement via projections from the underlying brainstem region. Through this the cerebellum acts as a comparator device to adjust fine motor control by monitoring a given limb movement in comparison to what cortical commands have requested. All sensory input arrives either through mossy fibers or climbing fibers (inferior olive), but by far the largest number of sensory inputs arrive through mossy fibers that synapse in the granule cell layer. There is also an established history of the importance of synaptic plasticity in mediating motor learning, with a central focus on long-term depression (LTD) at the parallel fiber to Purkinje cell synapse. Work over the years however established multiple forms of plasticity at virtually all synaptic junctions in cerebellum²⁶. Finally, recent work has shifted attention to cerebellum in finding that motor functions are primarily mediated in the rostral half of cerebellum (lobules 1-5) while caudal lobules (6-10) are instead involved in processing input for cognitive functions²⁷.

Most of the work on Fragile X has centered on cortical or hippocampal circuits and the effects that a loss of FMRP can have on synaptic plasticity, leading to the mGluR theory of Fragile X²⁸. In this case work on LTD at the CA1 hippocampal synapse led to the overall proposal that a loss of FMRP leads to upregulation of mGluR (glutamate) receptors and the second messenger “extracellular signal-regulated kinase” (ERK). Other work has begun to highlight cerebellum as another focus for problems in Fragile X and cognitive dys-function. Selective knockout of FMRP in Purkinje cells, the primary output neuron of cerebellar cortex, revealed a role for FMRP in enhancing parallel fiber LTD and reducing the conditional eye-blink response that depends on cerebellar Purkinje cells²⁹. Multiple studies have documented structural changes in Purkinje cells²⁹ or hypovolemia in the midline region of cerebellum in patients with Fragile X^{30,31} or ASD³²⁻³⁴. The importance of disrupted sensory processing to both Fragile X and the expression of autism spectrum disorders (ASD) is increasingly recognized³⁵⁻³⁸. The cerebellum is also recognized to contribute to Fragile X syndrome through its role in monitoring sensory input related to cognitive functions that can contribute to ASD in Fragile X patients^{29,31,39-45}. A recent review posed that ASD behavioural symptoms could reflect the role of cerebellum in modifying the postnatal development of cortical synaptic circuitry and plasticity through its strong reciprocal connections with cortex that are shaped through the late development of cerebellum⁴⁶.

At the cellular level the loss of FMRP in Purkinje cells was tied to disruption of LTD of parallel fiber input that arises from granule cells^{29,31,39-44}. There are multiple synaptic junctions and forms of synaptic plasticity that have been analyzed for disruption in Fragile X beyond mossy fiber input to cerebellum⁴⁷. We thus recognize that the extent to which signal processing at this particular synapse contributes to overall behavioural changes upon administering tat-FMRP is currently unknown.

### The Role of FMRP in Neuronal Function

FMRP has been known to regulate transcription of a large number of mRNAs (called the FMRP transcriptome), miRNAs and other nuclear and cytosolic proteins. It can thus regulate translation of proteins through actions that range from transcriptional control in the nucleus to ribosomal translation of proteins⁴⁸. FMRP can also differentially regulate the level of mRNA and proteins, with the balance of shift often depending on the number of CGG repeats present. Thus, individuals who have a substantial number of CGG repeats (30-200) are identified as FMRP carriers, or can develop motor control problems in FMRP-ataxia later in life^{39,40,49-52}. In some cases the level of mRNA can increase dramatically in Fragile X without a corresponding increase in protein levels^{49,53}. We are studying the effects of a complete loss of FMRP, in which Fragile X patients can exhibit profound reduction in cognitive abilities (IQ 40-70), hyperactivity, disruption in social interactions and ASD, and in many cases hyper aggression^{48,54}. Many of these behaviours are successfully reproduced in an animal model of Fragile X in which fmrp1 transcription is entirely prevented in a transgenic line of FMRP−/− mice. Since this is an X chromosome-linked trait all of our studies have focused on male mice of P16-90 days of age, ranging from a period of late development (ie adolescent) to full adult.

A large body of work has documented how FMRP regulates the levels of proteins available to mediate changes in synaptic strength needed for cognitive functions, which is central to the mGluR theory of Fragile X. These typically involve changes in the levels of transmitter receptors or second messengers important to regulating receptor trafficking or phosphorylation important to synaptic plasticity. More recently reports have emerged of the ability for FMRP to regulate the activity of voltage- or calcium-gated ion channels that control cell excitability.

FMRP and Ion channels: Analyses of the brain FMRP transcriptome have revealed a large number of mRNAs that translate proteins for ion channels⁵⁵⁻⁵⁸. In brainstem synaptosomes FMRP was coimmunoprecipitated with Kv3.1b mRNA. In addition to binding with Kv3.1b mRNA, FMRP regulation of Kv3.1b protein expression was found in the brainstem sound localization circuit 59. In another set of studies FMRP was shown to interact with Kv4.2 mRNA in hippocampus^{60,61}, which constitutes the major component of A-type potassium current in pyramidal neurons. The absence of FMRP in FMRP−/− mice decreased Kv4.2 mRNA translation and protein expression levels 60. A similar reduction of Kv4.2 protein level was detected in cortical homogenates, implicating positive regulation by FMRP on Kv4.2 expression. However, the findings of another study questioned the exact regulatory role of FMRP on Kv4.2 expression levels. Lee et al. 2011⁶¹ found the opposite effect of FMRP suppressing Kv4.2 expression levels in hippocampal neurons⁶¹, with ˜1.5-2 fold increase of Kv4.2 protein expression observed in hippocampal regions of fmr1-KO mice compared to the wt counterparts. The reason for these opposing effects was not defined although the use of two different mouse strains might provide an explanation.

The newest data reveals that FMRP can also directly interact with select ion channels. FMRP was found to coimmunoprecipitate with Slack potassium channels in synaptosomes prepared from mouse olfactory bulb and brain stem regions⁵⁶. FMRP further coimmunoprecipitates with Slack-B channels in exogenously expressed HEK cells, with FMRP binding to the Slack-B C-terminus. Given that FMRP acts to increase activation of Slack potassium channels, a reduced Slack current was observed in FMRP−/− mice. This group was also responsible for identifying the 1-298 aa segment of the FMRP N terminus as harboring an N-terminal protein-protein interaction domain (NDF) that influenced Slack-B channel gating⁵⁶. The molecule has since been made commercially available (Novus Biology) and is the construct we have tested against the calcium channel Cav3.1 and potassium channel Kv4.3.

In another set of studies, FMRP was found to modulate big conductance calcium-activated potassium channel (BK) function and gating characteristics^{62,63}. FMRP was found to modulate BKCa channel functions by directly interacting with the β accessory subunit. Loss of FMRP in FMRP−/− mice led to excessive broadening of action potential duration and enhanced presynaptic calcium influx in hippocampal and cortical neurons⁶². Single channel BK channel analysis in CA3 pyramidal neurons revealed a reduction of channel open probability in FMRP−/− mice⁶³.

In addition to modulatory roles of FMRP on potassium channel function, FMRP can control the membrane density of N-type (Cav2.2) voltage-gated calcium channels in dorsal root ganglion (DRG) neurons⁵⁸. A loss of FMRP protein with shRNA knockdown increased Cav2.2 channel density at the somatic cell sur-face and at the presynaptic terminals of DRG neurons. Expression of FMRP in tsA-201 cells reduced the Cav2.2 current density by decreasing channel expression at the plasma membrane, with no change in Cav2.2 gating characteristics⁵⁸. Interactions between FMRP and Cav2.2 channels at the C-terminal and II-III linker regions were shown through coimmunoprecipitation⁵⁸.

One distinction to be made is that the C-terminal domain of FMRP interacts with Cav2.2 channels, whereas FMRP interactions with Slack-B and the BK-β4 subunit involve the N-terminal half of FMRP. While this indicates that our use of a tat-FMRP(298) fragment of the N-terminus could modulate more ion channels than just the Cav3-Kv4 complex, it also predicts that it should not affect the density of Cav2.2 calcium channels, a channel involved in regulating transmitter release.

### Treatment Strategies for Fragile X Syndrome

Given the background knowledge of molecular mechanisms of FMRP and its regulation of protein levels, a growing number of potential therapies are being pursued^{65, 66}. These can be broadly grouped into targeting disruptions in synaptic receptor activation, second messengers activated typically by mGluRs, and molecular approaches to reduce the number of CGG repeats or its actions on fmrp1 transcription.

mGluR signaling: The mGluR theory of Fragile X syndrome rests on the premise that a loss of FMRP leads to disrupted levels of proteins required for synaptic plasticity^{28,67}. Of 8 forms of mGluR receptors, the most pertinent to Fragile X syndrome belong to the Group 1 family that includes mGluR1 and mGluR5 isoforms. The loss of FMRP in FXS results in an excitatory-inhibitory imbalance and disruptions in long term plasticity identified in specific cortical and cerebellar neurons 68. For instance, the expression levels of mGluR1 is altered in cortex but not hippocampus or cerebellum in FMRP−/− mice⁴. LTD in the CA1 hippocampal region is enhanced through a loss of FMRP downregulation of proteins involved in AMPAR receptor internalization^{69,70}. The involvement of mGluR1 or mGluR5 isoforms is region-specific but both shown to influence behaviours in Fragile X syndrome. An increase in mGluR1-dependent LTD is found at the cerebellar parallel fiber-Purkinje cell synapse in FMRP−/− animals²⁹. mGluR5 disruption is known to modify cortical functions and a range of behaviours in Fragile X syndrome⁷¹⁻⁷⁴.

The number of mGluR-related synaptic functions disrupted by a loss of FMRP has led to multiple studies on the ability to pharmacologically reduce aberrant behaviours in Fragile X syndrome⁷⁵. Administration of the mGluR1 antagonist JNJ16259685 has been shown to correct repetitive behaviour and to mildly improve seizure susceptibility, but with no apparent effect on motor function in the context of PPI or rotarod performance⁷⁶. Genetic reduction of mGluR5 or pharmacological block ameliorates a broad array of Fragile X symptoms, including over activity in the ERK and mTOR signalling pathways, repetitive behaviour, audiogenic seizures, and disrupted prepulse inhibition^{68,76-83}.

ERK signaling pathway: The mGluR theory of FXS includes a role for phosphorylated extracellular signal regulated kinase (ERK)²⁸. ERK1/2 are an important subclass of the mammalian mitogen-activated protein kinase (MAPK) family of serine/threonine kinases and play important roles in the regulation of learning, memory and behaviour^{84,85}. mGluR-mediated LTD in hippocampus relies on activation of ERK by phos-phorylation (pERK) and the increased levels of protein found in Fragile X Syndrome^{86,87}. The loss of FMRP in Fragile X syndrome has been reported to elevate basal levels of pERK in both Fragile X patients and mouse models⁸⁸⁻⁹⁰. Yet others found that FMRP−/− mice exhibit dephosphorylation of ERK following mGluR1/5 stimulation⁹¹. The direction of change in pERK can also vary according to brain region. Finally, other groups report no obvious elevation of pERK but instead a hypersensitivity of the ERK signaling path-way to upstream signals^{86,92}.

The extent to which ERK is involved in disrupting circuit function and behaviours in Fragile X is thus still an ongoing question. Regardless, the availability of clinically approved ERK blockers has led to clinical studies that report some success in restoring levels of pERK in FXS patients. By example, lovastatin, a hypocholesterolemic drug, reduces ERK-mediated functions by decreasing activation of its upstream component Ras^{13,14,87,88}. Lovastatin normalizes excessive protein synthesis in the hippocampus of FMRP−/− mice and prevents mGluR-induced epileptogenesis⁸⁶. In patients lovastatin returned the levels of ERK activation to normal with improved cognition and adaptive behavior^{13,88}. Most recently, the drug Metformin, already approved for use in treating diabetes, was shown to ameliorate deficits in Fragile X by affecting the MEK-ERK pathway and the eIF4E signal^{17,90}.

Molecular restoration of fmrp1 transcription: Several strategies have been tested and are still underway to restore translation of FMRP at the genetic level. Reintroducing FMRP expression by AAV viral transfection had great promise, but was offset by variability in the distribution and expression levels of FMRP, in which overexpression proved to be toxic or even fatal^{43,93-97}.

Other groups are testing the means to reduce the number of CGG repeats in the untranslated region that disrupts fmrp1 gene transcription. These studies are in an early stage in focusing on fmrp1-expressing hybrid cell lines or human (FXS) pluripotent stem cells, but establish that CRISPR/Cas9 gene editing that removes some or all of the CGG repeats can restore transcription and FMRP production^{98,99}. The extent to which this occurs, however, depends on factors related to relieving the extent of hypermethylation of the fmr1 gene, and potentially even more than the CGG repeats per se^{20,99-101}.

Tat-FMRP as a therapeutic agent: Delivering proteins or drugs to CNS neurons must contend with the presence of a blood-brain barrier (BBB) formed primarily by endothelial cells that line the vessels of the cerebrovasculature. Several strategies are being tested to act cell penetrating peptides to deliver drugs across the BBB that take advantage of endogenous transport pathways, passive or active carrier-mediated transport, or transcytosis (for reviews 105-109). One of the most well defined methods at this time is the use of a short segment of HIV-1 regulatory protein, trans-activator of transcription (tat) peptide^{23-25,106}. The ability to use a tat-FMRP approach was assessed earlier and concluded the approach was not efficient in transfer across the BBB or into CNS neurons, and was toxic above a specific level¹¹⁰. However, this study used a full length FMRP as a tat conjugate.

The above summary documents progress being made on several key fronts to either restore FMRP translation or reduce the effects of disrupted receptor-mediated activation of second messenger pathways. However, the genetic modification strategy is still at an early stage of in vitro assessment, while the complexity and number of proteins deregulated upon loss of FMRP makes a receptor or second-messenger targeted strategy open to innumerable issues of target specificity or compensation.

Our data can be grouped into showing progress on three fronts on how:
1) FMRP interacts with two new ion channels (Cav3.1 and Kv4.3) to regulate their amplitude and functions on the basis of shifts in voltage dependence and/or membrane density
2) FMRP uses the Cav3-Kv4 complex to produce a postsynaptic change in intrinsic excitability of cerebellar granule cells to produce LTP of mossy fiber input to shape sensory processing
3) Tat-FMRP(298) can reduce aberrant behavioural traits inherent to FMRP−/− mice within 1 hour of tail vein injection by crossing the blood brain barrier to enter a vast array of central neurons
4) Tat-FMRP(298) produces no signs of toxicity over 24 hr exposure (100 nM) in dissociated granule cell cultures.

Cav3-Kv4 at the Mossy fiber-granule cell synapse: Mossy fiber input reflects the largest source of sensory input to cerebellar granule cells before information is sent to Purkinje cells, the output cell of the cerebellar cortex. As such, the mossy fiber-granule cell synaptic junction reflects a functional gateway to the cerebellar cortex where synaptic plasticity shapes sensory input. The ability to induce LTP at this synapse has long been recognized and to be active even in vivo in response to physiological patterns of sensory stimulation¹¹¹⁻¹¹³. Any dysfunction at the mossy fiber-granule cell synaptic junction will thus impair signal processing by the cerebellar cortex at the first stage of sensory input.

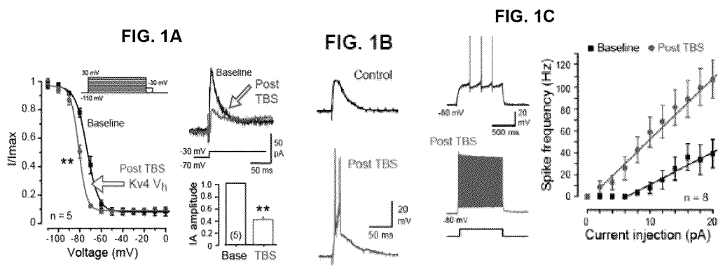

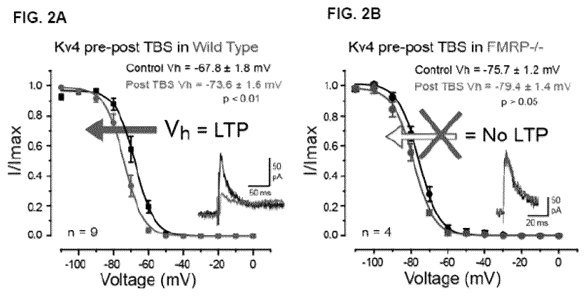

Our work on Fragile X syndrome centers on an ion channel complex in which the voltage-gated Kv4 potassium channel gains calcium-dependent modulation by associating with Cav3 (T-type) calcium channels¹¹⁴⁻¹¹⁸. Our previous work established that the normal role for a Cav3-Kv4 complex in cerebellar neurons is to increase A-type potassium current amplitude to decrease excitability and spike output¹¹⁴⁻¹¹⁸. The Cav3-Kv4 complex also proves to be highly sensitive to any change in Cav3 conductance, such that a decrease in calcium influx reduces A-type current by shifting the voltage dependence for Kv4 channels in a negative direction (termed here as a “left-shift in Kv4 Vh”)^{114 117}. We recently found that the Cav3-Kv4 complex in granule cells is also involved in producing LTP of the mossy fiber-evoked postsynaptic response through a similar process. Here a theta burst pattern of mossy fiber input produces a long-lasting l eft-shift in Kv4 Vh to reduce A-type current and enhance granule cell excitability (FIG. 1)¹¹⁸. This is important in revealing a strong postsynaptic component to LTP by modulating the intrinsic excitability of a neuron through an identified ion channel complex that we can test.

FMRP in mossy fiber synaptic function: We predicted that the Cav3-Kv4 complex will be relevant to Fragile X Syndrome in that loss of FMRP affects synaptic plasticity in cerebellar and cortical regions^{4,7,115,116,119-125}, and the known ability for FMRP to regulate at least Kv4.2 potassium channels^{126,127}. We also know that a strong functional coupling between Cav3 and Kv4 channels allows factors that affect Cav3 channels to be imparted on Kv4 channels to alter A-type current amplitude¹¹⁴⁻¹¹⁸. Our preliminary data have returned the surprising result that FMRP is a member of the Cav3-Kv4 complex and is required for potentiation mediated by this complex. Specifically, FMRP regulates membrane excitability of granule cells by associating with Cav3.1 channels within the Cav3-Kv4 complex. The key role for FMRP at this synapse was confirmed by our findings that mossy fiber LTP and a reduction in A-type current are both absent in FMRP−/− mice, the direct model of Fragile X syndrome. Moreover, introducing an active fragment of FMRP rein-states the capacity for mossy fiber synaptic plasticity, and even reduces behavioural dysfunction in adult FMRP−/− mice. Details on the data supporting these conclusions are shown below.

### Background/Data

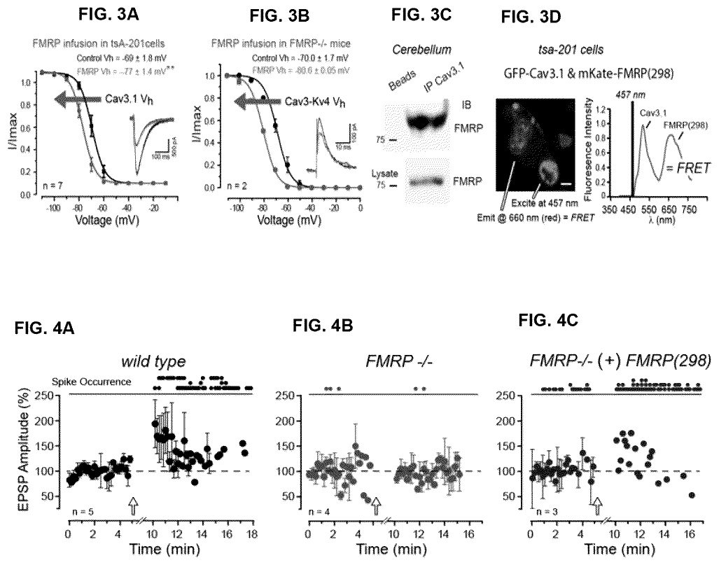

LTP at the mossy fiber synapse is produced by a measureable shift in the voltage dependence of A-type current measured under whole-cell recording conditions in granule cells maintained in a slice preparation in vitro (FIG. 3). The parameter tested is thus referred to as a “left shift in Kv4 Vh”, which reduces Kv4 channel availability and A-type potassium current (see inset). It is also important to note that given the tight coupling between Cav3.1 and Kv4 channels in the complex, any changes in Cav3.1 calcium channel voltage-dependence or availability will also be conferred onto that of Kv4 channels. When Kv4 current is isolated for study in granule cells we do not block Cav3 channels, allowing us to measure what is referred to as the “Cav3-Kv4 Vh”.

Our data reveal that a theta-burst stimulus delivered to mossy fibers that evokes a left shift in Kv4 Vh in wt mice (FIG. 3A) fails to do so in FMRP−/− animals (FIG. 3B). To test the ability for FMRP to reverse this result we intracellularly infused a short active N-terminal fragment (aa 1-298) of FMRP¹²⁸ through the re-cording electrode. Given the influence of Cav3 channel properties on A-type current we first tested this on Cav3.1 channels expressed in isolation in tsA-201 cells. Here infusing 30 nM FMRP(298) evoked a left shift in Cav3.1 Vh that reduced T-type calcium current (FIG. 4A). We also found that infusing 30 nM FMRP(298) into granule cells of FMRP−/− mice left-shifted the Cav3-Kv4 Vh (FIG. 4B). In support of a role for Cav3 channels we find that FMRP coimmunoprecipitates (coIPs) with Cav3.1 from cerebellar lysates (FIG. 4C). Moreover, coexpressing GFP-Cav3.1 and mKate-FMRP fluorophore-tagged constructs in tsA-201 cells reveals Foerster Resonance Energy Transfer (FRET) (FIG. 4D). These data reveal a very close association be-tween Cav3.1 and FMRP, as donor-acceptor proteins must be positioned at <10 nm distance to satisfy the requirements to achieve FRET.

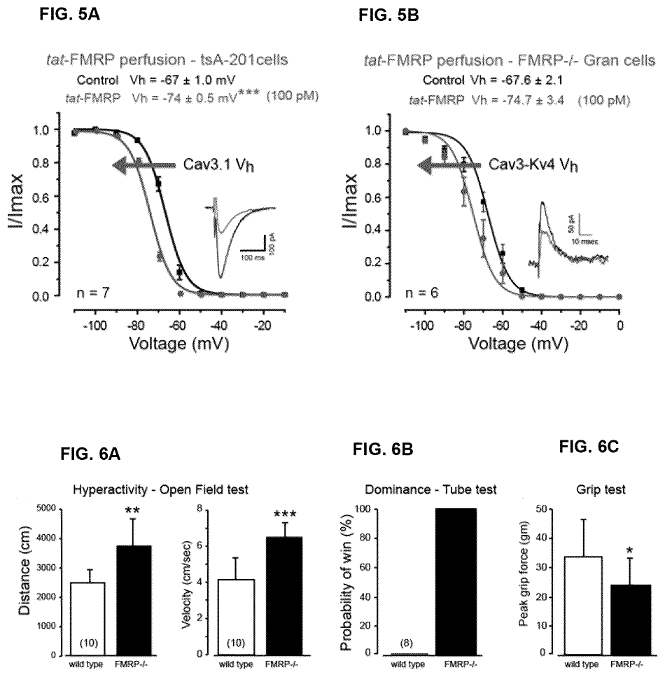

In an initial test on the role of FMRP in mossy fiber LTP, theta burst stimulation of mossy fibers in wt mice potentiated the EPSP and increased spike discharge (FIG. 5A). However, the same stimulus in FMRP−/− mice failed to increase EPSP amplitude or spike firing (FIG. 5B). Infusing FMRP(298) into cells then re-stored the ability for theta burst stimuli to potentiate the EPSP and increase spike firing (FIG. 5C).

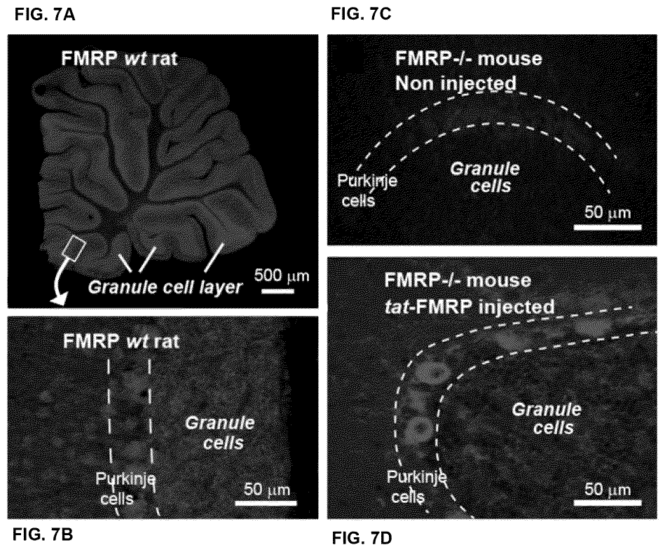

Behavioural tests: We conducted a first set of tests on P60-90 wt animals and a small group of FMRP−/− mice. In an Open Field experiment, a common test conducted on Fragile X mice for hyperactivity¹²⁹, we found that FMRP−/− mice exhibited significantly higher velocity and distance traveled (FIG. 6A) than wt mice over 30 min. In a test of social dominance we used a Tube test in which animals are allowed to enter a tube from either end to determine which animal is able to force a counterpart back out of the end of the tube. Here FMRP−/− mice proved to win every contest, suggesting a higher level of social dominance or aggression than wt mice (FIG. 7B). Finally, in a test of grip strength (a common cerebellar-motor related task), FMRP−/− mice showed a weaker peak grip force than wt animals (FIG. 6C). These tests are important as initial evidence that FMRP−/− mice differ from wt animals on several behavioural traits that we can use to test the efficacy of replacing FMRP.

tat-FMRP(298): To implement tests of FMRP infusion at a whole animal level we developed a tat construct of the shorter N-terminal fragment FMRP(298)¹²⁸ previously shown to modulate Slack ion channels. To prepare tat-FMRP(298) we cloned the fragment into a pTrcHis vector containing a tat and His peptide sequence and expressed the cDNA in BL21 pLysS E coli to generate tat-FMRP(298) protein. The final tat-FMRP(298) construct is 33 kDa, a size that is within the range for high efficiency transport¹³⁰. Initial tests applying tat-FMRP(298) at 100 pM in the external medium in vitro established that it rapidly produced a left-shift of Cav3.1 Vh in tsA-201 cells (FIG. 5A) similar to that produced by internally infused 35 nM FMRP(298) (cf FIG. 3A). A similar test conducted in FMRP−/− granule cells revealed a significant left-shift in the Cav3-Kv4 Vh upon bath application of 100 pM tat-FMRP(298) (FIG. 5B). These tests are important in establishing that FMRP(298) successfully incorporates as a tat peptide to penetrate the cell membrane and retain its activity on the Cav3-Kv4 complex.

A series of tests have now been conducted to assess the ability to use this tat construct as a means of gaining access to central neurons in vivo. A major potential hurdle is to ensure that a peripheral administered compound can pass the blood-brain barrier and achieve effective penetration of neurons in the CNS. In the case of FMRP this process does not need to be selective, in that FMRP is almost ubiquitously expressed in both neurons and glia over all brain regions. We are thus interested in achieving as widespread a pattern of introducing FMRP as possible. We first conducted tests to define FMRP expression in cerebellum using rats of FVB/S129 mice prepared for immunocytochemistry to identify immunolabel indicated by an antibody against the N-terminal region of FMRP (Novus Biology). These tests establish that FMRP is widely distributed and expressed in all major cell types identified thus far, including granule cells, Purkinje cells, and both basket and stellate cells in the molecular layer (FIG. 7A, B). By comparison, there is no detectable FMRP immunolabel in cerebellum of FMRP−/− mice processed in the same manner (FIG. 7C). But after 1 hr of injecting tat-FMPR(298) injected into the tail vein of FMRP−/− mice at 100 nM concentration, brains processed for immunocytochemistry show that FMRP immunolabel delivered through this tat construct is pre-sent within the same cells as found in wt animals (FIG. 7D). These data verify a high efficiency of tat-FMRP(298) transport across the blood-brain barrier, and widespread uptake by cerebellar neurons after tail vein injection.

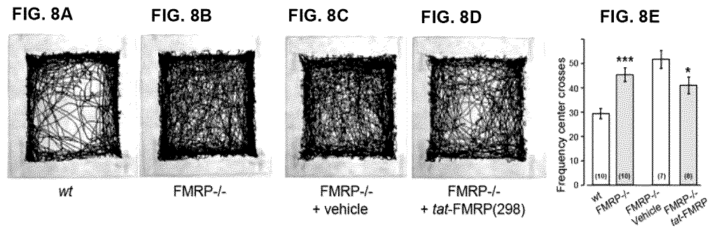

The key test was to determine if any behavioural traits of FMRP−/− could be reduced by tat-FMRP(298) administration. Here the Open Field test confirmed that FMRP−/− mice showed evidence of hyperactivity by exhibiting significantly higher frequencies of crossing the center region of a cage compared to wt mice (FIG. 8A, B, E). However, FMRP−/− mice that received a tail vein injection of 100 nM tat-FMRP(298) within only 1 hour showed a significantly lower frequency of crossings compared to FMRP−/− mice that received vehicle injection (FIG. 8C-E). These results are exciting in providing an initial proof of concept that tat-FMRP(298) can induce a measureable change in the behaviour of FMRP−/− mice. An interesting aspect of these results is that behavioural modification was achieved in mice 2-3 months old, suggesting its ability to modify Fragile X-related behaviours even in adult animals, and within only 1 hour of delivery in the periphery.

METHODS: We used FMRP−/− and wt mice bred on the FVB/129 background (JAX) given a reported high prevalence of autistic-like symptoms¹³¹⁻¹³⁸. Whole-cell recordings are obtained in granule cells of cerebellar vermis in tissue slices in vitro, dissociated granule cell cultures^{117,118,139}, or in tsA-201 cells expressing subunits of the Cav3-Kv4 complex¹¹⁴⁻¹¹⁸. To measure Kv4 current postsynaptically and maintain excitatory synaptic inputs we externally apply 2 mM CsCl, 5 mM TEA, and 50 μM picrotoxin, and internally apply 5 mM TEA and 0.1 QX-314 to block HCN, sodium and non-Kv4 potassium channels117,118, with internal patch solutions described in Rizwan et al.¹¹⁸. tsA-201 recordings will focus on Cav3.1 channels as this iso-form exhibits the highest expression level in granule cells¹¹⁷. Dissociated cerebellar granule cell cultures will be prepared using previously reported procedures¹⁴⁰. CoIPs, pull-down assays onto GST fusion proteins, and immunocytochemistry will follow previous reports^{115,116,118}. Fluorophore-tagged constructs for FRET will be prepared and tested on a spectral confocal microscope¹⁴¹.

We use the ALA 2PK+ Pipette perfusion system^{118,139} to internally infuse FMRP(298)¹²⁸ (Novus Biology) through the patch electrode dissolved in (mM): 50 NaH₂PO₄, 300 NaCl, 250 imidazole, pH 8.0, applied at ≤3 nM. tat-FMRP(298) is bath applied in vitro or in a saline carrier medium by tail vein injection in iso-fluorane anesthetized mice to achieve a final plasma concentration of 100 nM. Immunocytochemistry is per-formed on free-floating tissue sections prepared after cardiac perfusion of paraformaldehyde and tissue preparation as detailed in previous reports.

### Example 2

In the following Example, the following experiments that have extended the original findings by adding additional data.

### Effects of FMRP(1-298) Infusion on Kv4 Current

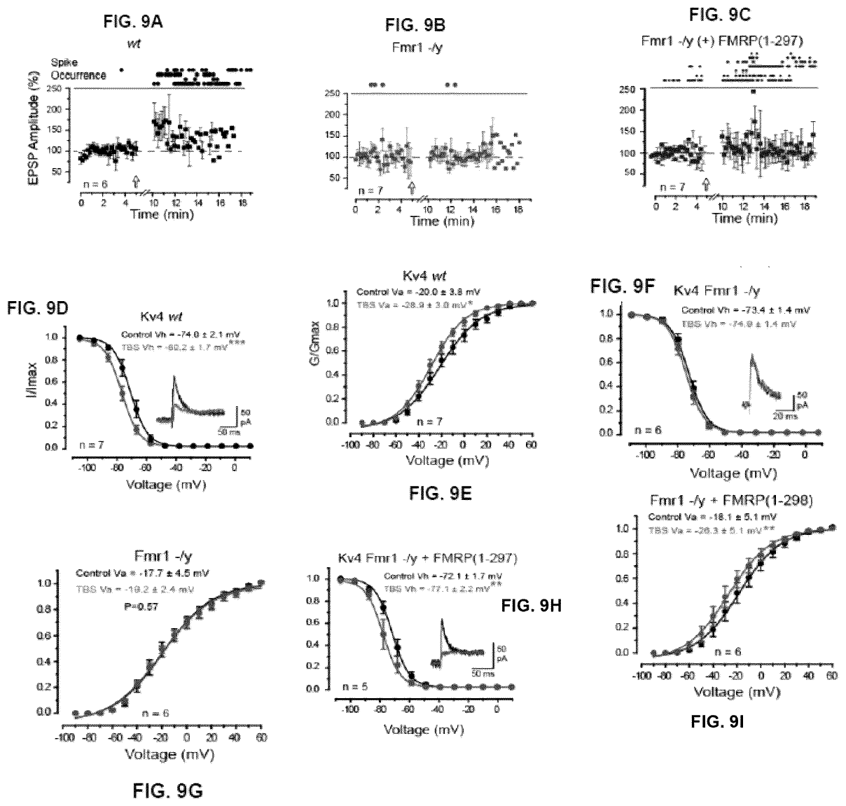

In FIG. 9 we added measurements of the effects of FMRP(1-298) infusion on the voltage for activation of Kv4 current (FIG. 9E, G, I). While the left shift in voltage for half activation (Vh) could potentially increase Kv4 current activation, the accompanying left-shift in half inactivation (Vh) voltage (FIG. 9D, F, H) produces a net decrease of Kv4 current in granule cells.

### tat-FMRP Testing In Vivo

### Concentration-Dependent Effects on Hyperactivity of Live Animals

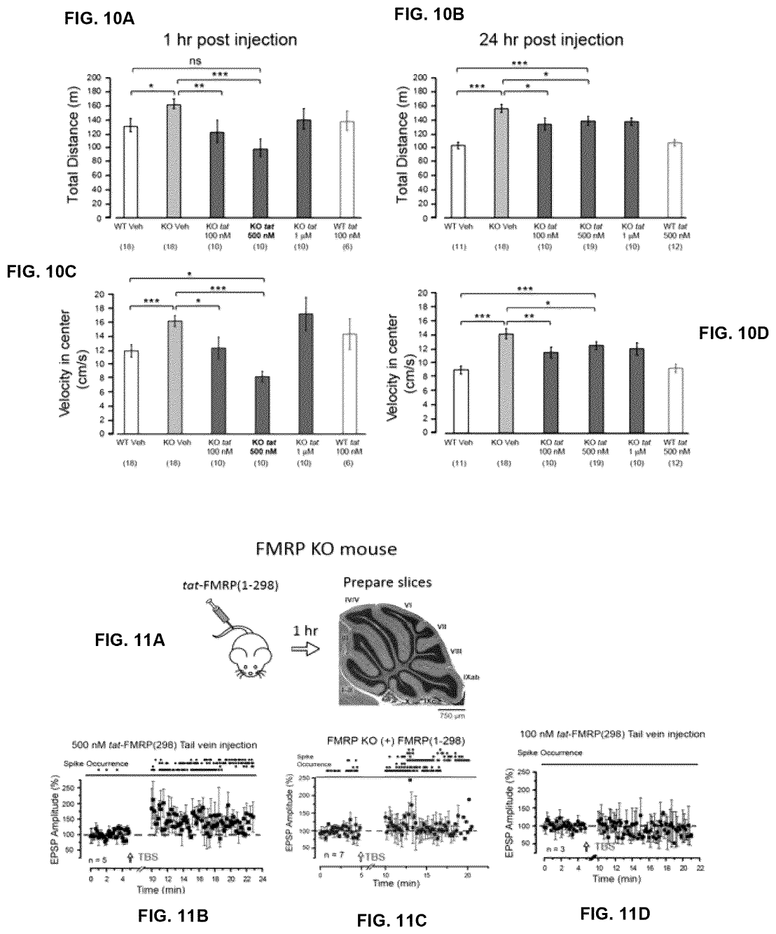

We initially had data that 100 nM tat-FMRP(1-298) injected into the tail of FMRP KO mice would alleviate some aspects of hyperactivity in the Open Field test (OFT). We extended this by carrying out OFT tests on P25, P40 and P60 animals. The data showed that the largest reduction in hyperactivity was obtained in P60-P80 animals, which forms the focus of the rest of the study. Here we found that certain aspects of hyperactivity were reduced by 100 nM, but even more by 500 nM tat-FMRP(1-298) injections when tested 1 hr after injections (FIG. 10). These effects could then be detected at reduced levels 24 hr after injections. However, the effects of tat-FMRP(1-298) were reduced or reversed for 1 microM injections (FIG. 10), suggesting that this concentration exceeds the healthy dose to use to reduce hyperactivity.

### tat-FMRP(1-298) Testing In Vitro

We had initially established that infusing 3-30 nM concentration of FMRP(1-298) directly into granule cells could partially restore LTP at the mossy fiber-granule cell synapse in vitro. After testing the effects of tail vein injected tat-FMRP(1-298) on the open field test (OFT) of behaving animals we injected FMRP KO animals with 500 nM tat-FMRP(1-298) and then prepared tissue slices 1 hr later to test its effects on restoring LTP (FIG. 11B). Here we found that injections of 500 nM tat-FMRP(1-298) restored LTP at the cellular level (FIG. 11B) to an even greater extent than direct injections of 3 nM tat-FMRP(1-298) previously conducted (FIG. 11C). This was apparent in a recovery of both the EPSP amplitude and increase in firing frequency after mossy fiber stimulation (FIG. 11B) compared to primarily an effect on spike frequency by direct infusion (Fig. C). These data are important in suggesting that delivery of this molecule by tail vein injection is even more effective than direct cellular infusion. Moreover, tail vein injections of 100 nM tat-FMRP(1-298) did not restore LTP at the mossy fiber synapse of FMRP KO animals (FIG. 11D), despite the ability to reduce aspects of hyperactivity in the live animal (FIG. 10). It would thus appear that behavioural effects of tat-FMRP(1-298) at 100 nM concentration include factors beyond just plasticity at the mossy fiber synapse, a result that was not entirely unexpected. The greater effects detected at the behavioural level with 500 nM tat-FMRP(1-298) are reproduced in vitro however, indicating a clear correlate between the concentration-dependent effects of tat-FMRP(1-298) tail vein injections on behavior, and the influence of this compound at the cellular level on a form of synaptic plasticity relevant to signal processing.

### Toxicity

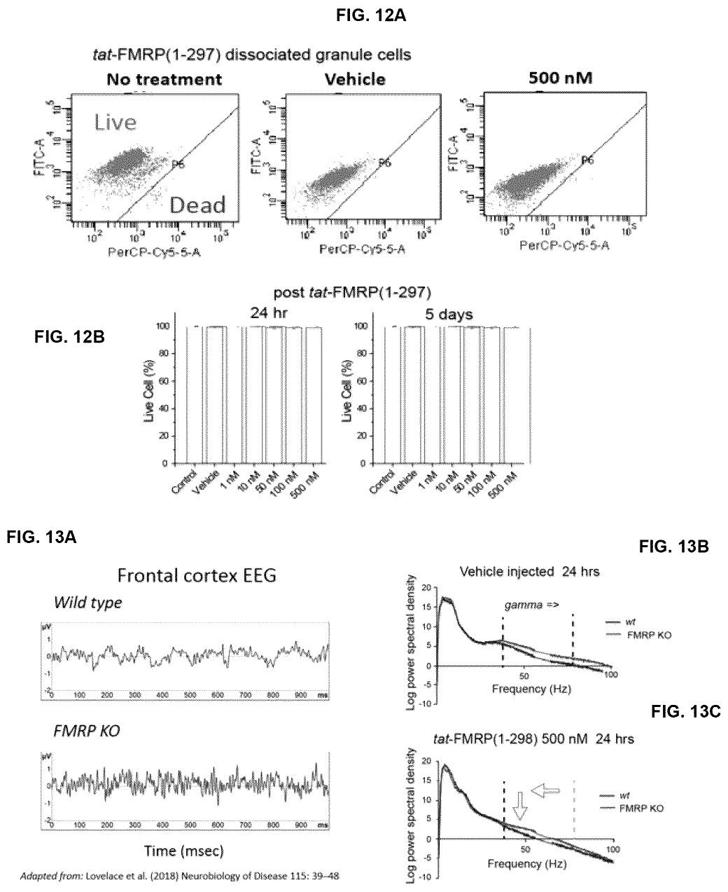

To test the potential toxicity of tat-FMRP(1-298) we prepared dissociated cultures of granule cells and delivered a single dose of vehicle alone or different concentrations of tat-FMRP(1-298). Cells were then tested at either 24 hrs or 5 days later following lysing exposure to reagents of a live-dead cell kit to measure through flow cytometry (FIG. 12). These tests showed no toxicity of tat-FMRP(1-298) even at the high dose of 500 nM (expected to be far higher than that attained in vivo) up to 5 days later.

### EEG Recordings

We extended this work to recording EEG, a measure of electrical activity across a much wider area of the brain, to test injections of tat-FMRP(1-298) ability to exert influence across the whole brain. A recent study reported that FMRP KO mice exhibit an increased level of EEG activity in the gamma frequency range (>40 Hz), a form of elevated resting activity that could interfere with sensory processing 42. We repeated this test and confirmed higher gamma frequency activity in FMRP KO mice compared to wt animals injected with only vehicle (FIG. 13A). We have thus far injected animals with 500 nM tat-FMRP(1-298), with analysis to date at 24 hrs post injection. Even these early measurements reveal a significant reduction in EEG gamma frequencies, and substantially more reduction in FMRP KO mice by tat-FMRP(1-298) (n=3) (FIG. 13B, C).

### REFERENCES

1. Khandjian, E.W. (1999). Biology of the fragile X mental retardation protein, an RNA-binding protein. Biochem Cell Biol 77(4): 331-42.
2. Dury, A.Y., et al. (2013). Nuclear Fragile X Mental Retardation Protein is localized to Cajal bodies. PLoS Genet 9(10): e1003890.
3. Zhao, M.G., et al. (2005). Deficits in trace fear memory and long-term potentiation in a mouse model for fragile X syndrome. J Neurosci 25(32): 7385-92.
4. Li, J., et al. (2002). Reduced cortical synaptic plasticity and GluR1 expression associated with fragile X mental retardation protein deficiency. Mol Cell Neurosci 19(2): 138-51.
5. Mazroui, R., et al. (2002). Trapping of messenger RNA by Fragile X Mental Retardation protein into cytoplasmic granules induces translation repression. Hum Mol Genet 11(24): 3007-17.
6. Shang, Y., et al. (2009). Fragile X mental retardation protein is required for chemically-induced long-term potentiation of the hippocampus in adult mice. J Neurochem 111(3): 635-46.
7. Yau, S.Y., et al. (2016). Impaired bidirectional NMDA receptor dependent synaptic plasticity in the dentate gyrus of adult female Fmr1 heterozygous knockout mice. Neurobiol Dis 96: 261-270.
8. Sidorov, M.S., et al. (2013). Fragile X mental retardation protein and synaptic plasticity. Mol Brain 6: 15.
9. Ferron, L., et al. (2014). Fragile X mental retardation protein controls synaptic vesicle exocytosis by modulating N-type calcium channel density. Nat Commun 5: 3628.
10. Verkerk, A.J., et al. (1991). Identification of a gene (FMR-1) containing a CGG repeat coincident with a breakpoint cluster region exhibiting length variation in fragile X syndrome. Cell 65(5): 905-14.
11. Papale, A., et al. (2016). Impairment of cocaine-mediated behaviours in mice by clinically relevant Ras-ERK inhibitors. Elife 5.
12. Uehling, D.E., et al. (2015). Recent progress on MAP kinase pathway inhibitors. Bioorg Med Chem Lett 25(19): 4047-56.
13. Caku, A., et al. (2014). Effect of lovastatin on behavior in children and adults with fragile X syndrome: an open-label study. Am J Med Genet A 164A(11): 2834-42.
14. Osterweil, E.K., et al. (2013). Lovastatin corrects excess protein synthesis and prevents epileptogenesis in a mouse model of fragile X syndrome. Neuron 77(2): 243-50.
15. Lovelace, J.W., et al. (2016). Matrix metalloproteinase-9 deletion rescues auditory evoked potential habituation deficit in a mouse model of Fragile X Syndrome. Neurobiol Dis 89: 126-35.
16. Gkogkas, C.G., et al. (2014). Pharmacogenetic inhibition of eIF4E-dependent Mmp9 mRNA translation reverses fragile X syndrome-like phenotypes. Cell Rep 9(5): 1742-55.
17. Gantois, I., et al. (2017). Metformin ameliorates core deficits in a mouse model of fragile X syndrome. Nat Med 23(6): 674-677.
18. Yau, S.Y., et al. (2016). Chronic minocycline treatment improves social recognition memory in adult male Fmr1 knockout mice. Behav Brain Res 312: 77-83.
19. Aguilar-Valles, A., et al. (2015). Inhibition of Group I Metabotropic Glutamate Receptors Reverses Autistic-Like Phenotypes Caused by Deficiency of the Translation Repressor eIF4E Binding Protein 2. J Neurosci 35(31): 11125-32.
20. Chiurazzi, P., et al. (1998). In vitro reactivation of the FMR1 gene involved in fragile X syndrome. Hum Mol Genet 7(1): 109-13.
21. Michalon, A., et al. (2014). Chronic metabotropic glutamate receptor 5 inhibition corrects local alterations of brain activity and improves cognitive performance in fragile X mice. Biol Psychiatry 75(3): 189-97.
22. Schaefer, T.L., et al. (2015). Emerging pharmacologic treatment options for fragile X syndrome. Appl Clin Genet 8: 75-93.
23. Zhao, M., et al. (2004). Intracellular cargo delivery using tat peptide and derivatives. Med Res Rev 24(1): 1-12.
24. Sawant, R., et al. (2010). Intracellular transduction using cell-penetrating peptides. Mol Biosyst 6(4): 628-40.
25. Wadia, J.S., et al. (2003). Modulation of cellular function by TAT mediated transduction of full length proteins. Curr Protein Pept Sci 4(2): 97-104.
26. Schonewille, M., et al. (2011). Reevaluating the role of LTD in cerebellar motor learning. Neuron 70(1): 43-50.
27. Stoodley, C.J., et al. (2009). Functional topography in the human cerebellum: a meta-analysis of neuroimaging studies. Neuroimage 44(2): 489-501.
28. Bear, M.F., et al. (2004). The mGluR theory of fragile X mental retardation. Trends Neurosci 27(7): 370-7.
29. Koekkoek, S.K., et al. (2005). Deletion of FMR1 in Purkinje cells enhances parallel fiber LTD, enlarges spines, and attenuates cerebellar eyelid conditioning in Fragile X syndrome. Neuron 47(3): 339-52.
30. Steinlin, M. (2008). Cerebellar disorders in childhood: cognitive problems. Cerebellum 7(4): 607-10.
31. Huber, K.M. (2006). The fragile X-cerebellum connection. Trends Neurosci 29(4): 183-5.
32. Hove, M.J., et al. (2015). Postural sway and regional cerebellar volume in adults with attention-deficit/hyperactivity disorder. Neuroimage Clin 8: 422-8.
33. Khan, A.J., et al. (2015). Cerebro-cerebellar Resting-State Functional Connectivity in Children and Adolescents with Autism Spectrum Disorder. Biol Psychiatry 78(9): 625-34.
34. Skefos, J., et al. (2014). Regional alterations in purkinje cell density in patients with autism. PLoS One 9(2): e81255.
35. Chen, L., et al. (2001). Fragile X mice develop sensory hyperreactivity to auditory stimuli. Neuroscience 103(4): 1043-50.
36. Arnett, M.T., et al. (2014). Deficits in tactile learning in a mouse model of fragile X syndrome. PLoS One 9(10): e109116.
37. Berzhanskaya, J., et al. (2016). Sensory hypo-excitability in a rat model of fetal development in Fragile X Syndrome. Sci Rep 6: 30769.
38. Sinclair, D., et al. (2016). Sensory processing in autism spectrum disorders and Fragile X syndrome-From the clinic to animal models. Neurosci Biobehav Rev.
39. Foote, M., et al. (2016). Fragile X-Associated Tremor/Ataxia Syndrome (FXTAS) Motor Dysfunction Modeled in Mice. Cerebellum 15(5): 611-22.
40. Hagerman, P.J., et al. (2015). Fragile X-associated tremor/ataxia syndrome. Ann N Y Acad Sci 1338: 58-70.
41. O'Keefe, J.A., et al. (2015). Characterization and Early Detection of Balance Deficits in Fragile X Premutation Carriers With and Without Fragile X-Associated Tremor/Ataxia Syndrome (FXTAS). Cerebellum 14(6): 650-62.
42. Pacey, L.K., et al. (2015). Persistent astrocyte activation in the fragile X mouse cerebellum. Brain Behav 5(10): e00400.
43. Gholizadeh, S., et al. (2014). Reduced phenotypic severity following adeno-associated virus-mediated Fmr1 gene delivery in fragile X mice. Neuropsychopharmacology 39(13): 3100-11.
44. Hampson, D.R., et al. (2015). Autism spectrum disorders and neuropathology of the cerebellum. Front Neurosci 9: 420.
45. D'Mello, A.M., et al. (2015). Cerebro-cerebellar circuits in autism spectrum disorder. Front Neurosci 9: 408.
46. Wang, S.S., et al. (2014). The cerebellum, sensitive periods, and autism. Neuron 83(3): 518-32.
47. Bostrom, C.A., et al. (2015). Rescue of NMDAR-dependent synaptic plasticity in Fmr1 knock-out mice. Cereb Cortex 25(1): 271-9.
48. Santoro, M.R., et al. (2012). Molecular mechanisms of fragile X syndrome: a twenty-year perspective. Annu Rev Pathol 7: 219-45.
49. Berman, R.F., et al. (2014). Mouse models of the fragile X premutation and fragile X-associated tremor/ataxia syndrome. J Neurodev Disord 6(1): 25.
50. Berman, R.F., et al. (2009). Mouse models of fragile X-associated tremor ataxia. J Investig Med 57(8): 837-41.
51. Botta-Orfila, T., et al. (2016). Molecular Pathophysiology of Fragile X-Associated Tremor/Ataxia Syndrome and Perspectives for Drug Development. Cerebellum 15(5): 599-610.
52. Brown, S.S., et al. (2015). Fragile X premutation carriers: A systematic review of neuroimaging findings. J Neurol Sci 352(1-2): 19-28.
53. Todd, P.K., et al. (2013). CGG repeat-associated translation mediates neurodegeneration in fragile X tremor ataxia syndrome. Neuron 78(3): 440-55.
54. Wheeler, A.C., et al. (2016). Aggression in fragile X syndrome. J Intellect Disabil Res 60(2): 113-25.
55. Brager, D.H., et al. (2014). Channelopathies and dendritic dysfunction in fragile X syndrome. Brain Res Bull 103: 11-7.
56. Brown, M.R., et al. (2010). Fragile X mental retardation protein controls gating of the sodium-activated potassium channel Slack. Nat Neurosci 13(7): 819-21.
57. Darnell, J.C., et al. (2011). FMRP stalls ribosomal translocation on mRNAs linked to synaptic function and autism. Cell 146(2): 247-61.
58. Ferron, L. (2016). Fragile X mental retardation protein controls ion channel expression and activity. J Physiol 594(20): 5861-5867.
59. Strumbos, J.G., et al. (2010). Fragile X mental retardation protein is required for rapid experience-dependent regulation of the potassium channel Kv3.1b. J Neurosci 30(31): 10263-71.
60. Gross, C., et al. (2011). Fragile X Mental Retardation Protein Regulates Protein Expression and mRNA Translation of the Potassium Channel Kv4.2. Journal of Neuroscience 31(15): 5693-5698.
61. Lee, H.Y., et al. (2011). Bidirectional regulation of dendritic voltage-gated potassium channels by the fragile X mental retardation protein. Neuron 72(4): 630-42.
62. Deng, P.Y., et al. (2013). FMRP regulates neurotransmitter release and synaptic information transmission by modulating action potential duration via BK channels. Neuron 77(4): 696-711.
63. Deng, P.Y., et al. (2016). Genetic upregulation of BK channel activity normalizes multiple synaptic and circuit defects in a mouse model of fragile X syndrome. J Physiol 594(1): 83-97.
64. BRANDALISE, F., et al. Cell-type specific regulation of ion channel function by Fragile X mental retardation protein. in Proc Soc Neurosci. 2017. Washington DC.
65. Levenga, J., et al. (2010). Potential therapeutic interventions for fragile X syndrome. Trends Mol Med 16(11): 516-27.
66. Erickson, C.A., et al. (2017). Fragile X targeted pharmacotherapy: lessons learned and future directions. J Neurodev Disord 9: 7.
67. Huber, K.M., et al. (2000). Role for rapid dendritic protein synthesis in hippocampal mGluR-dependent long-term depression. Science 288(5469): 1254-7.
68. Bagni, C., et al. (2013). Fragile X syndrome: From protein function to therapy. Am J Med Genet A 161A(11): 2809-21.
69. Park, S., et al. (2008). Elongation factor 2 and fragile X mental retardation protein control the dynamic translation of Arc/Arg3.1 essential for mGluR-LTD. Neuron 59(1): 70-83.
70. Waung, M.W., et al. (2008). Rapid translation of Arc/Arg3.1 selectively mediates mGluR-dependent LTD through persistent increases in AMPAR endocytosis rate. Neuron 59(1): 84-97.
71. Ronesi, J.A., et al. (2012). Disrupted Homer scaffolds mediate abnormal mGluR5 function in a mouse model of fragile X syndrome. Nat Neurosci 15(3): 431-40, s1.
72. Guo, W., et al. (2016). Selective Disruption of Metabotropic Glutamate Receptor 5-Homer Interactions Mimics Phenotypes of Fragile X Syndrome in Mice. J Neurosci 36(7): 2131-47.
73. Dolen, G., et al. (2008). Role for metabotropic glutamate receptor 5 (mGluR5) in the pathogenesis of fragile X syndrome. J Physiol 586(6): 1503-8.
74. Hays, S.A., et al. (2011). Altered neocortical rhythmic activity states in Fmr1 KO mice are due to enhanced mGluR5 signaling and involve changes in excitatory circuitry. J Neurosci 31(40): 14223-34.
75. Scharf, S.H., et al. (2015). Metabotropic glutamate receptor 5 as drug target for Fragile X syndrome. Curr Opin Pharmacol 20: 124-34.
76. Thomas, A.M., et al. (2012). Group I metabotropic glutamate receptor antagonists alter select behaviors in a mouse model for fragile X syndrome. Psychopharmacology (Berl) 219(1): 47-58.
77. Yan, Q., et al. (2005). Suppression of two major Fragile X Syndrome mouse model phenotypes by the mGluR5 antagonist MPEP. Neuropharmacology 49(7): 1053-1066.
78. de Vrij, F.M., et al. (2008). Rescue of behavioral phenotype and neuronal protrusion morphology in Fmr1 KO mice. Neurobiology of disease 31(1): 127-132.
79. Dolen, G., et al. (2007). Correction of fragile X syndrome in mice. Neuron 56(6): 955-62.
80. Tatarczynska, E., et al. (2001). Potential anxiolytic- and antidepressant-like effects of MPEP, a potent, selective and systemically active mGlu5 receptor antagonist. Br J Pharmacol 132(7): 1423-30.
81. Yan, Q.J., et al. (2005). Suppression of two major Fragile X Syndrome mouse model phenotypes by the mGluR5 antagonist MPEP. Neuropharmacology 49(7): 1053-66.
82. Busquets-Garcia, A., et al. (2013). Targeting the endocannabinoid system in the treatment of fragile X syndrome. Nat Med 19(5): 603-7.
83. Michalon, A., et al. (2012). Chronic pharmacological mGlu5 inhibition corrects fragile X in adult mice. Neuron 74(1): 49-56.
84. Peng, S., et al. (2010). ERK in Learning and Memory: A Review of Recent Research. International Journal of Molecular Sciences 11(1): 222-232.
85. Fasano, S., et al. (2011). Ras-ERK signaling in behavior: old questions and new perspectives. Frontiers in Behavioral Neuroscience 5.
86. Osterweil, E.K., et al. (2010). Hypersensitivity to mGluR5 and ERK1/2 leads to excessive protein synthesis in the hippocampus of a mouse model of fragile X syndrome. J Neurosci 30(46): 15616-27.
87. Banko, J.L., et al. (2006). Regulation of eukaryotic initiation factor 4E by converging signaling pathways during metabotropic glutamate receptor-dependent long-term depression. J Neurosci 26(8): 2167-73.
88. Pellerin, D., et al. (2016). Lovastatin corrects ERK pathway hyperactivation in fragile X syndrome: potential of platelet's signaling cascades as new outcome measures in clinical trials. Biomarkers 21(6): 497-508.
89. Hou, L., et al. (2006). Dynamic translational and proteasomal regulation of fragile X mental retardation protein controls mGluR-dependent long-term depression. Neuron 51(4): 441-54.
90. Wang, X., et al. (2012). Activation of the extracellular signal-regulated kinase pathway contributes to the behavioral deficit of fragile x-syndrome. J Neurochem 121(4): 672-9.
91. Kim, S.H., et al. (2008). Aberrant early-phase ERK inactivation impedes neuronal function in fragile X syndrome. Proc Natl Acad Sci U S A 105(11): 4429-34.
92. Liu, Z.H., et al. (2012). Lithium reverses increased rates of cerebral protein synthesis in a mouse model of fragile X syndrome. Neurobiol Dis 45(3): 1145-52.
93. Arsenault, J., et al. (2016). FMRP Expression Levels in Mouse Central Nervous System Neurons Determine Behavioral Phenotype. Hum Gene Ther 27(12): 982-996.
94. Gholizadeh, S., et al. (2015). Expression of fragile X mental retardation protein in neurons and glia of the developing and adult mouse brain. Brain Res 1596: 22-30.
95. Zeier, Z., et al. (2009). Fragile X mental retardation protein replacement restores hippocampal synaptic function in a mouse model of fragile X syndrome. Gene Ther 16(9): 1122-9.
96. Gantois, I., et al. (2001). Restoring the phenotype of fragile X syndrome: insight from the mouse model. Curr Mol Med 1(4): 447-55.
97. SIEGEL, J., et al. Restoration of FMRP in the prefrontal cortex of adult Fragile X mice post-development rescues prefrontal-associated deficits in Proc Soc Neuroscience. 2017. Washington DC.
98. Park, C.Y., et al. (2015). Reversion of FMR1 Methylation and Silencing by Editing the Triplet Repeats in Fragile X iPSC-Derived Neurons. Cell Rep 13(2): 234-41.
99. Xie, N., et al. (2016). Reactivation of FMR1 by CRISPR/Cas9-Mediated Deletion of the Expanded CGG-Repeat of the Fragile X Chromosome. PLoS One 11(10): e0165499.
100. Bar-Nur, O., et al. (2012). Molecular analysis of FMR1 reactivation in fragile-X induced pluripotent stem cells and their neuronal derivatives. J Mol Cell Biol 4(3): 180-3.
101. Tabolacci, E., et al. (2016). Transcriptional Reactivation of the FMR1 Gene. A Possible Approach to the Treatment of the Fragile X Syndrome. Genes (Basel) 7(8).
102. ROTH, M., et al. Development of a sensitive and quantitative assay to detect FMRP in cell lines and human tissues. in Proc Soc Neurosci. 2017. Washington DC: FULCRUM Therapeutics, Cambridge, MA.
103. WU, H., et al. Quantitative assessment of the contribution of FMR1 to function in iPSC-derived Fragile X neurons. in Proc Soc Neurosci. 2017. Washington DC.
104. GRAEF, J.D., et al. Functional assessment of spontaneous and evoked activity in iPSC-derived Fragile X neurons using multielectrode array (MEA) and fluorometric imaging plate reader (FLIPR) platforms. in Proc Soc Neurosci. 2017. Washington DC.
105. Oller-Salvia, B., et al. (2016). Blood-brain barrier shuttle peptides: an emerging paradigm for brain delivery. Chem Soc Rev 45(17): 4690-707.
106. Rizzuti, M., et al. (2015). Therapeutic applications of the cell-penetrating HIV-1 Tat peptide. Drug Discov Today 20(1): 76-85.
107. Foged, C., et al. (2008). Cell-penetrating peptides for drug delivery across membrane barriers. Expert Opin Drug Deliv 5(1): 105-17.
108. Kurrikoff, K., et al. (2016). Recent in vivo advances in cell-penetrating peptide-assisted drug delivery. Expert Opin Drug Deliv 13(3): 373-87.
109. Bolhassani, A., et al. (2017). In vitro and in vivo delivery of therapeutic proteins using cell penetrating peptides. Peptides 87: 50-63.
110. Reis, S.A., et al. (2004). Prospects of TAT-mediated protein therapy for fragile X syndrome. J Mol Histol 35(4): 389-95.
111. D'Angelo, E. (2005). Synaptic plasticity at the cerebellum input stage: mechanisms and functional implications. Arch Ital Biol 143(2): 143-56.
112. Gandolfi, D., et al. (2015). Long-Term Spatiotemporal Reconfiguration of Neuronal Activity Revealed by Voltage-Sensitive Dye Imaging in the Cerebellar Granular Layer. Neural Plast 2015: 284986.
113. Roggeri, L., et al. (2008). Tactile stimulation evokes long-term synaptic plasticity in the granular layer of cerebellum. J Neurosci 28(25): 6354-9.
114. Anderson, D., et al. (2013). The Cav3-Kv4 complex acts as a calcium sensor to maintain inhibitory charge transfer during extracellular calcium fluctuations. J Neurosci 33(18): 7811-24.
115. Anderson, D., et al. (2010). Regulation of neuronal activity by Cav3-Kv4channel signaling complexes. Nat Neurosci 13(3): 333-7.
116. Anderson, D., et al. (2010). Regulation of the KV4.2 complex by CaV3.1 calcium channels. Channels (Austin) 4(3): 163-7.
117. Heath, N.C., et al. (2014). The expression pattern of a Cav3-Kv4 complex differentially regulates spike output in cerebellar granule cells. J Neurosci 34(26): 8800-12.
118. Rizwan, A.P., et al. (2016). Long-term potentiation at the mossy fiber-granule cell relay invokes postsynaptic second-messenger regulation of Kv4 channels. J Neurosci 36(44): 11196-11207.
119. Deng, P.Y., et al. (2011). Abnormal presynaptic short-term plasticity and information processing in a mouse model of fragile X syndrome. J Neurosci 31(30): 10971-82.
120. Desai, N.S., et al. (2006). Early postnatal plasticity in neocortex of Fmr1knockout mice. J Neurophysiol 96(4): 1734-45.
121. Huber, K.M., et al. (2002). Altered synaptic plasticity in a mouse model of fragile X mental retardation. Proc Natl Acad Sci U S A 99(11): 7746-50.
122. Piochon, C., et al. (2014). Cerebellar plasticity and motor learning deficits in a copy-number variation mouse model of autism. Nat Commun 5: 5586.
123. Mckay, B.E., et al. (2006). Ca(V)3 T-type calcium channel isoforms differentially distribute to somatic and dendritic compartments in rat central neurons. Eur J Neurosci 24(9): 2581-94.
124. Rudy, B., et al. (1992). Region-specific expression of a K+ channel gene in brain. Proc Natl Acad Sci U S A 89(10): 4603-7.
125. Serodio, P., et al. (1998). Differential expression of Kv4 K+ channel subunits mediating subthreshold transient K+ (A-type) currents in rat brain. J Neurophysiol 79(2): 1081-91.
126. Gross, C., et al. (2011). Fragile X mental retardation protein regulates protein expression and mRNA translation of the potassium channel Kv4.2. J Neurosci 31(15): 5693-8.
127. Spencer, K.B., et al. (2016). FMRP Mediates Chronic Ethanol-Induced Changes in NMDA, Kv4.2, and KChIP3 Expression in the Hippocampus. Alcohol Clin Exp Res 40(6): 1251-61.
128. Zhang, Y., et al. (2012). Regulation of neuronal excitability by interaction of fragile X mental retardation protein with slack potassium channels. J Neurosci 32(44): 15318-27.
129. Kazdoba, T.M., et al. (2014). Modeling fragile X syndrome in the Fmr1 knockout mouse. Intractable Rare Dis Res 3(4): 118-33.
130. Leibrand, C.R., et al. (2017). HIV-1 Tat disrupts blood-brain barrier integrity and increases phagocytic perivascular macrophages and microglia in the dorsal striatum of transgenic mice. Neurosci Lett 640: 136-143.
131. Moy, S.S., et al. (2009). Social approach in genetically engineered mouse lines relevant to autism. Genes Brain Behav 8(2): 129-42.
132. Liu, Z.H., et al. (2009). Dissociation of social and nonsocial anxiety in a mouse model of fragile X syndrome. Neurosci Lett 454(1): 62-6.
133. Spencer, C.M., et al. (2011). Modifying behavioral phenotypes in Fmr1KO mice: genetic background differences reveal autistic-like responses. Autism Res 4(1): 40-56.
134. Rotschafer, S.E., et al. (2012). Minocycline treatment reverses ultrasonic vocalization production deficit in a mouse model of Fragile X Syndrome. Brain Res 1439 :7-14.
135. Lai, J.K., et al. (2014). Temporal and spectral differences in the ultrasonic vocalizations of fragile X knock out mice during postnatal development. Behav Brain Res 259: 119-30.
136. Dolan, B.M., et al. (2013). Rescue of fragile X syndrome phenotypes in Fmr1 KO mice by the small-molecule PAK inhibitor FRAX486. Proc Natl Acad Sci U S A 110(14): 5671-6.
137. Spencer, C.M., et al. (2005). Altered anxiety-related and social behaviors in the Fmr1 knockout mouse model of fragile X syndrome. Genes Brain Behav 4(7): 420-30.
138. Bilousova, T.V., et al. (2009). Minocycline promotes dendritic spine maturation and improves behavioural performance in the fragile X mouse model. J Med Genet 46(2): 94-102.
139. King, B., et al. (2015). IKCa channels are a critical determinant of the slow AHP in CA1 pyramidal neurons. Cell Rep 11(2): 175-82.
140. Zhan, X.Q., et al. (2014). Abeta40 modulates GABA (A) receptor alpha6 subunit expression and rat cerebellar granule neuron maturation through the ERK/mTOR pathway. J Neurochem 128(3): 350-62.
141. Asmara, H., et al. (2017). A T-type channel-calmodulin complex triggers alphaCaMKII activation. Mol Brain 10(1): 37.
142. Adinolfi, S., et al. (2003). The N-terminus of the fragile X mental retardation protein contains a novel domain involved in dimerization and RNA binding. Biochemistry 42(35): 10437-44.
143. Trehin, R., et al. (2004). Chances and pitfalls of cell penetrating peptides for cellular drug delivery. Eur J Pharm Biopharm 58(2): 209-23.
144. Sabatier, J.M., et al. (1991). Evidence for neurotoxic activity of tat from human immunodeficiency virus type 1. J Virol 65(2): 961-7.
145. Yun, S.W., et al. (2006). Fmrp is required for the establishment of the startle response during the critical period of auditory development. Brain Res 1110(1): 159-65.
146. Belagodu, A.P., et al. (2016). Characterization of ultrasonic vocalizations of Fragile X mice. Behav Brain Res 310: 76-83.
147. McNaughton, C.H., et al. (2008). Evidence for social anxiety and impaired social cognition in a mouse model of fragile X syndrome. Behav Neurosci 122(2): 293-300.
148. Sorensen, E.M., et al. (2015). Hyperactivity and lack of social discrimination in the adolescent Fmr1 knockout mouse. Behav Pharmacol 26(8 Spec No): 733-40.
149. Roy, S., et al. (2012). Comprehensive analysis of ultrasonic vocalizations in a mouse model of fragile X syndrome reveals limited, call type specific deficits. PLoS One 7(9): e44816.
150. Nielsen, D.M., et al. (2002). Alterations in the auditory startle response in Fmr1 targeted mutant mouse models of fragile X syndrome. Brain Res 927(1): 8-17.
151. Garcia-Caballero, A., et al. (2014). The deubiquitinating enzyme USP5 modulates neuropathic and inflammatory pain by enhancing Cav3.2 channel activity. Neuron 83(5): 1144-58.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A recombinant fusion polypeptide comprising or consisting of a cell penetrating polypeptide and a FMRP(298) polypeptide, or fragment or variants thereof.

2. The recombinant fusion polypeptide of claim 1, wherein said cell penetrating polypeptide comprises a tat polypeptide.

3. The recombinant fusion polypeptide of claim 2, wherein said tat polypeptide comprises YGRKKRRQRRR (SEQ ID NO: 2).

4. The recombinant fusion polypeptide of claim 1, further comprising a HIS polypeptide.

5. The recombinant fusion polypeptide of claim 4, wherein said HIS polypeptide comprises MGGSHHHHHHGMAS (SEQ ID NO: 3).

6. A fusion polypeptide comprising or consisting of tat-FMRP(298) MEELVVEVRGSNGAFYKAFVKDVHEDSITVAFENNWQPDRQIPFHDVRFPPPVGYNKDINESD EVEVYSRANEKEPCCWWLAKVRMIKGEFYVIEYAACDATYNEIVTIERLRSVNPNKPATKDTFH KIKLDVPEDLRQMCAKEAAHKDFKKAVGAFSVTYDPENYQLVILSINEVTSKRAHMLIDMHFRSL RTKLSLIMRNEEASKQLESSRQLASRFHEQFIVREDLMGLAIGTHGANIQQARKVPGVTAIDLDE DTCTFHIYGEDQDAVKKARSFLEFAEDVIQVPRNLVGKVIGSGGGYGRKKRRQRRR (SEQ ID NO: 1), or fragments or variants thereof.

7. The recombinant fusion polypeptide of claim 1, wherein said fusion polypeptide comprises a variant fusion polypeptide sequence that is at least 80-99% identical to said fusion polypeptide, or fragments or variants thereof.

8. The recombinant fusion polypeptide of claim 1, having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70,71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more than 100 amino acid substitutions.

9. A polynucleotide molecule comprising or consisting of a sequence that encodes a cell penetrating polypeptide and a FMRP(298) polypeptide, or fragment or variants thereof.

10. The polynucleotide molecule of claim 9, wherein said cell penetrating polypeptide comprises a tat polypeptide.

11. The polynucleotide molecule of claim 10, wherein said tat polypeptide comprises YGRKKRRQRRR (SEQ ID NO: 2).

12. A polynucleotide molecule comprising or consisting of a sequence that encodes a fusion polypeptide comprising or consisting of tat-FMRP(298) (SEQ ID NO: 1).

13. A polynucleotide molecule comprising or consisting of a sequence that encodes a fusion polypeptide according to claim 1.

14. A vector comprising the polynucleotide molecule of claim 9.

15. A mammalian cell comprising the polynucleotide molecule of claim 9.

16. A mammalian cell comprising the vector of claim 14.

17. A pharmaceutical composition comprising a recombinant fusion polypeptide of claim 1, and a pharmaceutically acceptable carrier.

18. A method of treatment of a subject having or suspected of having Fragile X Syndrome, comprising: administering a recombinant fusion polypeptide of claim 1 to said subject.

19. The method of claim 18, further comprising administration of minocycline, metformin, and/or blockers of extracellular signal-regulated kinase (ERK).

20. The method of claim 18, wherein said subject is a human.

21. A kit, comprising: a container; a recombinant fusion polypeptide of claim 1; and optionally instructions for the use thereof.
